(19) United States
(12) Patent Application Publication
(10) Pub. No.: US 2025/0387639 A1
(43) Pub. Date: Dec. 25, 2025

(54) FRACTIONAL HANDPIECE WITH A PASSIVELY Q-SWITCHED LASER ASSEMBLY WITH UNSTABLE CAVITY OPERATION

(71) Applicant: Candela Corporation, Marlborough, MA (US)

(72) Inventors: Xiaoming Shang, Lexington, MA (US); Kevin Schomacker, Maynard, MA (US); Yimin Zang, Marlborough, MA (US); Hugh Xiong, Marlborough, MA (US); Herb Otterson, Marlborough, MA (US)

(21) Appl. No.: 19/242,335

(22) Filed: Jun. 18, 2025

Related U.S. Application Data
(60) Provisional application No. 63/662,318, filed on Jun. 20, 2024.

(51) Int. Cl. A61N 5/06; H01S 3/102; H01S 3/1115; H01S 3/131

(52) U.S. Cl. CPC A61N 5/0616; H01S 3/1024; H01S 3/1115; H01S 3/131

(57) ABSTRACT

A fractional handpiece and systems thereof for skin treatment include a passively Q-switched laser assembly operatively connected to a pump laser source to receive a pump laser beam having a first wavelength and a beam-splitting assembly operable to scan a solid beam emitted by the passively Q-switched laser assembly and form an array of micro-beams across a segment of skin. The passively Q-switched laser assembly generates a sub-nanosecond pulsed laser beam having a second wavelength. The fractional handpiece may also incorporate a wavelength switching assembly, enabling selective delivery of both the second and third wavelengths, while providing control over the energy output at each wavelength, all within a single handpiece. The passively Q-switched laser assembly has an unstable cavity operation.

### FIELD

The present system relates to a passively Q-switched laser packaged in a handpiece and in particular to laser systems with a fractional handpiece with a passively Q-switched laser assembly and a beam-splitting assembly.

### BACKGROUND

Typically, systems for non-invasive treatment of skin disorders include a cabinet into which a laser is placed and a beam delivery system (typically an optical fiber or an articulated arm) connected to a handpiece that conducts the laser radiation from the laser to a segment of skin to be treated. The functionality of such a system is limited by the capabilities of the selected laser. Treatment of skin imperfections usually requires more than one type of laser and frequently more than one type of laser is placed in the cabinet. This increases size, cost and complexity of the system.

Treatment of some skin imperfections requires significant laser power (tens and even hundreds of MW) that in order to prevent skin damage is supplied in ultrashort pulses (most commonly in picosecond regime). Such laser power is difficult to transfer through a fiber and use of an articulated arm significantly limits the freedom of the caregiver.

A typical Q-switched microcavity laser consists of a laser medium and a saturable absorber as a passive Q-switcher positioned very close to each other. The cavity length is managed to be as short as possible. Q-switched microcavity lasers are small solid-state lasers with a linear short cavity. The typical cavity length is on the order of millimeters. The short cavity lengths result in extremely short cavity lifetimes, and the possibility of much shorter Q-switched pulses. It has been demonstrated that Q-switched microcavity lasers can produce output pulses on a sub-nanosecond regime. In some special cases (e.g., monolithic cavity), the pulse duration can be as short as large mode-locked lasers produce with peak powers of about 10KW, similar to commercially available large Q-switched systems produce.

Over decades, a lot of effort has been put in places striving for generation of high-energy picosecond lasers. Many techniques have been developed. These techniques commonly involve multi-stage configurations, e.g., a low-energy picosecond seed laser, for example nJ or μJ are fed into amplification stages (including regenerative amplifier or/and multi-pass amplifications). Such multi-stage configurations require complex optical arrangement and sophisticated electronic synchronization further increasing the complexity and cost of the system.

### BRIEF DESCRIPTION OF THE DRAWINGS

The patent or application file contains at least one drawing executed in color. Copies of this patent or patent application with color drawing(s) will be provided by the Office by request and payment of the necessary fee.

For a more complete understanding of this disclosure and its features, reference is now made to the following description, taken in conjunction with accompanying drawings, in which like reference numerals denote like elements:

FIG. 1 is an example of a system having a pump source, a pump laser delivery unit, and a sub-nanosecond fractional handpiece;

FIG. 2A is an example of a fractional handpiece with a passively Q-switched laser assembly and a beam-splitting assembly;

FIG. 2B is an example of a fractional handpiece with a pump laser source, a passively Q-switched laser assembly, and a beam-splitting assembly;

FIG. 2C is an example 1064 nm handpiece;

FIG. 2D is an example 532 nm handpiece;

FIG. 2E is an example 1064 nm handpiece with a homogenizer in the pump beam path;

FIG. 2F is an example 532 nm handpiece with a homogenizer in the pump beam path;

FIG. 3A is an example of a passively Q-switched laser assembly with a monolithic cavity;

FIG. 3B is an example of a monolithic cavity;

FIG. 3C is an example of a passively Q-switched laser assembly with a monolithic cavity and a homogenizer in the pump beam path;

FIG. 4 is an example illustrating the bonding of laser medium and saturable absorber of a microchip laser;

FIG. 5A is an example of a passively Q-switched laser assembly with a cavity with two external cavity mirrors;

FIG. 5B is an example of a passively Q-switched laser assembly with a cavity with one external cavity mirror (output coupler);

FIG. 5C is an example of a passively Q-switched laser assembly with a cavity with one external cavity mirror (high reflector);

FIG. 5D is an example of a passively Q-switched laser assembly with a cavity formed by one monolithic rod and two external cavity mirrors;

FIG. 5E is an example of a passively Q-switched laser assembly with a cavity formed by a monolithic rod and one external output coupler;

FIG. 5F is an example of a passively Q-switched laser assembly with a cavity formed by one monolithic rod and one external high reflector;

FIG. 5G is an example of a passively Q-switched laser assembly with a cavity with two external cavity mirrors and a homogenizer in the pump beam path;

FIG. 5H is an example of a passively Q-switched laser assembly with a cavity with one external cavity mirror (output coupler) and a homogenizer in the pump beam path;

FIG. 5I is an example of a passively Q-switched laser assembly with a cavity with one external cavity mirror (high reflector) and a homogenizer in the pump beam path;

FIG. 5J is an example of a passively Q-switched laser assembly with a cavity formed by one monolithic rod and two external cavity mirrors and a homogenizer in the pump beam path;

FIG. 5K is an example of a passively Q-switched laser assembly with a cavity formed by a monolithic rod and one external output coupler and a homogenizer in the pump beam path;

FIG. 5L is an example of a passively Q-switched laser assembly with a cavity formed by one monolithic rod and one external high reflector and a homogenizer in the pump beam path;

FIG. 5M is an example of a passively Q-switched laser assembly with an unstable resonator cavity formed by a plano high reflector and a convex output coupler;

FIG. 5N is an example of a passively Q-switched laser assembly with an unstable resonator cavity formed by a convex high reflector and a plano output coupler;

FIG. 5O is an example of a passively Q-switched laser assembly with an unstable resonator cavity formed by a coating on a concave surface of a bonded gain element and a plano output coupler;

FIG. 5P is an example of a passively Q-switched laser assembly with an unstable resonator cavity formed by a bonded gain element, a convex high reflector, and a coating on a distal end of a saturable absorber;

FIG. 5Q is an example of a passively Q-switched laser assembly with an unstable resonator cavity formed by a coating on a proximal end of a bonded element and a convex output coupler;

FIG. 5R is an example of a passively Q-switched laser assembly with an unstable resonator cavity formed by a bonded gain element, a convex high reflector, and a plano output coupler;

FIG. 5S is an example of a passively Q-switched laser assembly with an unstable resonator cavity formed by a bonded gain element, a plano high reflector, and a convex output coupler;

FIG. 5T is an example of a passively Q-switched laser assembly with an unstable resonator cavity formed by a coating on a proximal end of a concave bonded three-segment element and a coating on its distal end;

FIG. 5U is an example of a passively Q-switched laser assembly with an unstable resonator cavity formed by a convex high reflector and a coating on a distal end of a bonded three-segment element;

FIG. 5V is an example of a passively Q-switched laser assembly with an unstable resonator cavity formed by a convex output coupler and a coating on a proximal end of a bonded three-segment element;

FIG. 6 is an example of a fractional handpiece with a second harmonic generation assembly;

FIG. 7A is an example beam-splitting assembly;

FIG. 7B is an example output of a 1-D solid beam splitter and a resulting scan of the micro-dot array.

FIG. 7C is an example output of a 1-D donut beam splitter and a resulting scan of the micro-dot array.

FIG. 7D is an example of a 2-D microbeam pattern generated with the combination of a pair of scanning mirrors and a lens array.

FIG. 7E is an example of sub-nanosecond laser beams incident on a microlens array with a pair of scanning mirrors

FIG. 7F is an example of 2-D microbeam pattern generated with the setup shown in FIG. 7D.

FIG. 8A is an example of a sparse fractional skin treatment pattern;

FIG. 8B is an example of a denser fractional skin treatment pattern;

FIG. 8C is an example beam-splitting assembly with a fixed mirror and a scanning mirror;

FIG. 8D is an example beam-splitting assembly with two scanning mirrors;

FIG. 9A is an example of laser system with a fractional handpiece with a roller for fractional skin treatment;

FIGS. 9B and 9C are examples of a laser system with a fractional handpiece with a scanning mirror for fractional skin treatment;

FIG. 9D is an example of a laser system with a fractional handpiece with two scanning mirrors for fractional skin treatment;

FIG. 10A is an example of laser system with a fractional handpiece with second harmonic generation and a roller for fractional skin treatment;

FIGS. 10B-10D are examples of a fractional handpiece with a second harmonic generation assembly;

FIGS. 10E-10Q are examples of a fractional handpiece with a second harmonic generation assembly and a wavelength switching assembly;

FIG. 10R is an example of a moveable mirror having two different coated sections;

FIG. 10S is an example of a moveable mirror having two different coated sections;

FIG. 10T is an example of half-plate rotation verse pulse energy;

FIG. 11 is another example of a fractional handpiece for fractional skin treatment;

FIG. 12 is a flowchart of an exemplary method of skin treatment by delivering sub-nanosecond laser pulses to a patient in need thereof.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. The terms “comprising,” “including” and “having” are used interchangeably in this disclosure. The terms “comprising,” “including” and “having” mean to include, but not necessarily be limited to the things so described.

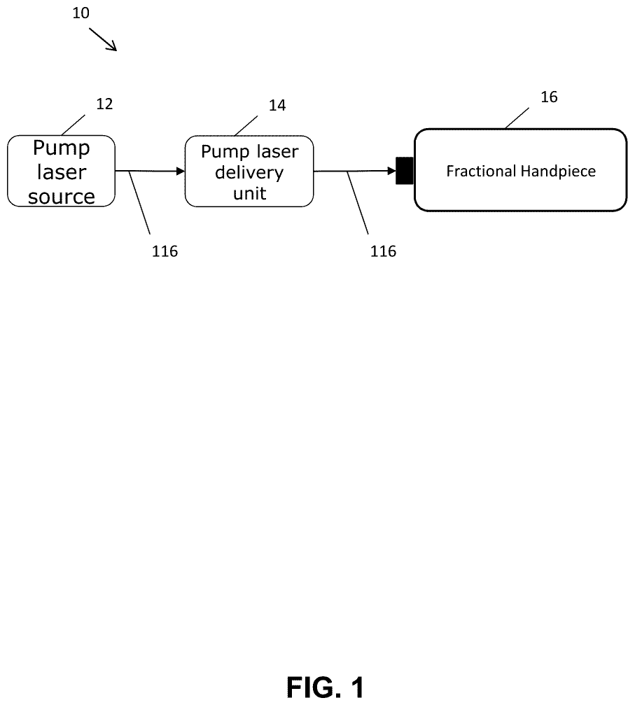

Disclosed herein is a system having a sub-nanosecond fractional handpiece with a passively Q-switched laser assembly and a method to implement the fractional handpiece. As shown in FIG. 1, the system 10 may include a pump laser source 12, a pump laser delivery unit 14, and the fractional handpiece 16.

In an example, the pump laser source 12 may be located in a cabinet. The pump laser source 12 may be any pump laser operable to provide energy to start the passively Q-switched laser in the handpiece to generate high-energy (>1 mJ) short pulses in a sub-nanosecond regime. For example, the pump laser may be operable to generate picosecond laser pulses with high peak power of about 100MW and higher when used in combination with the fractional handpiece. The pump laser source may be a laser emitting wavelength at which the laser rod has enough absorption. For example, for an Nd:YAG laser, the pump laser wavelength may be within one of four wavelength bands, e.g., 735-760 nm, 795-820 nm, or 865-885 nm. The pump laser may be a solid state laser or diode laser. Non-limiting examples of pump lasers include an Alexandrite laser (755 nm), a Ti:Sapphire laser, a diode laser, a dye laser, an optical parametric oscillator (OPO), and an optical parameter amplifier (OPA). Ti:Sapphire may be used to generate laser beams in the wavelength range between 700-900 nm via direct emission pumped in the visual wavelength region. In an example, an Alexandrite laser may provide over 1 kW pumping power for higher pulse energy generation. The high pumping power facilitates energy storage that is further facilitated by use of a saturable absorber of low initial transmission. The pump laser source 12 may operate at a single pulse up to a frequency of about 2000 Hz.

The pump laser delivery unit 14 may be operable to deliver the pump laser to the fractional handpiece for pumping the passively Q-switched laser. In some examples, the pump laser delivery unit may be an articulated arm which is an assembly of a number of mirrors and mechanical levers or arms connected between them by rotary joints. In an example, the articulated arm may have a plurality of arms (elbows) and a plurality of mirrors operable to direct the laser beam to a desired point on the fractional handpiece by rotation around at least one rotary joint connecting the plurality of arms. In an example, the plurality of mirrors is operable to preserve incident laser beam polarization, which may be useful for efficient pumping of anisotropic laser material (e.g., Nd:YAP and Nd:YLF). In additional examples, the pump laser delivery unity may include fiber optics and be delivered by an optical fiber. The optical fiber may a single mode fiber, multimode fiber, or hollow core fiber.

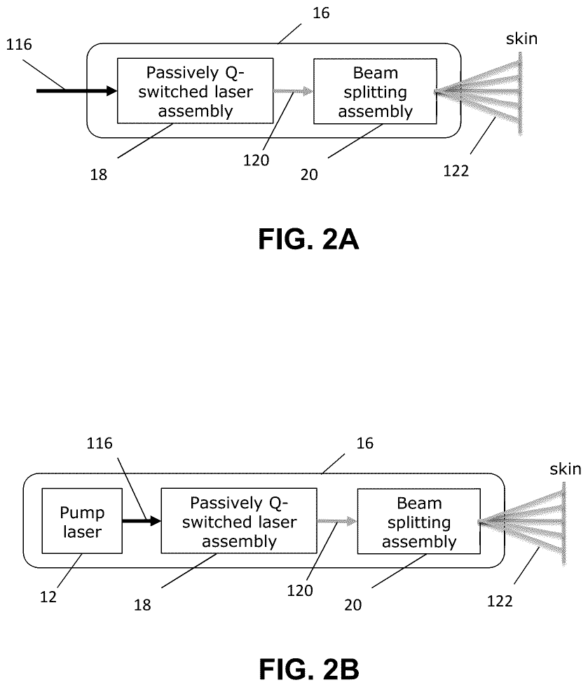

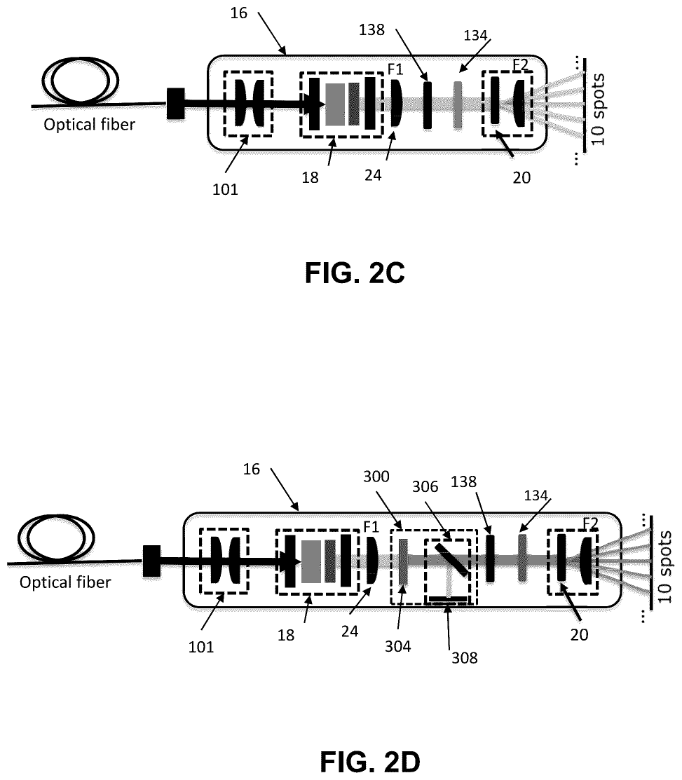

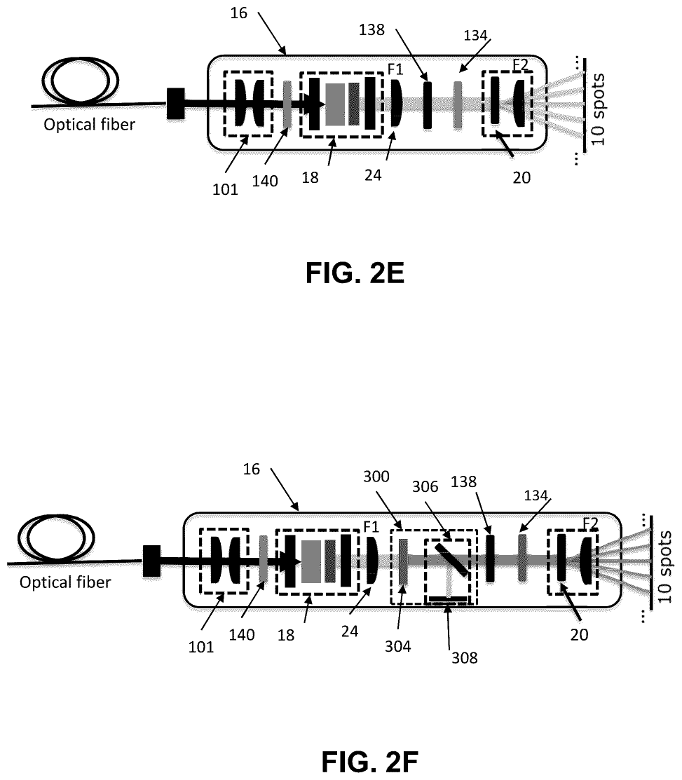

As seen in FIG. 2A, the fractional handpiece 16 may include a passively Q-switched laser assembly 18 and a beam-splitting assembly 20. The fractional handpiece is operable to generate high-energy (≥2 mJ) sub-nanosecond laser pulses and subsequently deliver those pulses to treatment sites (e.g., the skin) with a fractionated pattern. The fractional handpiece receives the pump laser delivered by the pump laser delivery unit to pump the passively Q-switched laser to generate high-energy (≥2 mJ) sub-nanosecond pulses. The generated sub-nanosecond laser is then split by the beam-splitting assembly into a microdot array that is delivered to the skin for fractional treatment. In some examples, the fractional handpiece may be operable to generate laser pulses with an energy of no less than 2 mJ. The fractional handpiece may not include an amplifier. In some examples, the beam-splitting assembly may have a repetition rate of about 100 pulses per second to about 500 pulses per second.

The dimensions of the passively Q-switched laser assembly allow it to be contained and mounted within the fractional handpiece body thereby reducing the size and complexity of the total system and improving the power utilization efficiency. The fractional handpiece may then be used in different applications and in particular for skin disorders treatment. The fractional handpiece body may be of a reasonable size and weight that easily fits within a user's hand and may be carried with a hand. The fractional handpiece may be less than or equal to 35 cm in length. In at least one example, the handpiece body may have a shape that facilitates it being held like a pencil. In other examples, the handpiece body may include a pistol grip that facilitates the handpiece body being held like a pistol.

In some examples, as seen in FIG. 2B, the pump laser source 12 and/or the pump laser delivery unit may also be small enough in size to be contained within the fractional handpiece body 16. In at least one example, the pump laser source 12 may be a diode laser that may be located within the fractional handpiece body and is operable to directly illuminate the passively Q-switched laser assembly.

FIG. 2C shows an example 1064 nm handpiece and FIG. 2D shows an example 532 nm handpiece, each using an optical fiber pump laser delivery unit. The fractional handpiece 16 in FIG. 2C includes pump lenses 101, a seed cavity with a passively Q-switched laser assembly 18, a collimating lens 24, a homogenizer 138, an attenuator 134, and a 1-D beam-splitting assembly 20. The fractional handpiece 16 in FIG. 2D includes pump lenses 101, a seed cavity with a passively Q-switched laser assembly 18, a collimating lens 24, a second harmonic generation assembly 300, a homogenizer 138, an attenuator 134, and a 1-D beam-splitting assembly 20.

FIG. 2E shows an example 1064 nm handpiece and FIG. 2F shows an example 532 nm handpiece, each using an optical fiber pump laser delivery unit and having a homogenizer in the pump beam path before the passively Q-switched laser assembly. The fractional handpiece 16 in FIG. 2E includes pump lenses 101, a first homogenizer 140, a seed cavity with a passively Q-switched laser assembly 18, a collimating lens 24, a second homogenizer 138, an attenuator 134, and a 1-D beam-splitting assembly 20. The fractional handpiece 16 in FIG. 2F includes pump lenses 101, a first homogenizer 140, a seed cavity with a passively Q-switched laser assembly 18, a collimating lens 24, a second harmonic generation assembly 300, a second homogenizer 138, an attenuator 134, and a 1-D beam-splitting assembly 20. The second homogenizer 138 may be diffractive-based or refractive-based. The second homogenizer 138 may facilitate mitigating beam characteristic variation at different repetition rates and homogenizing a beam profile delivered to the skin. It is important to note that for beam splitting based on a refractive or diffractive beam-splitter, the energy output from the passively Q-switched assembly must be sufficiently high to ensure that each microbeam generated after splitting retains adequate energy for effective skin treatment. For instance, if a treatment requires each microbeam to have 3 mJ of energy and 10 microbeams are split from an incoming beam, the passively Q-switched laser should produce no less than 30 mJ of energy. However, when beam splitting relies on scanning mirrors, each pulse from the passively Q-switched laser yields only one microbeam. In this scenario, the laser assembly only need to generate no less than 3 mJ of energy.

The passively Q-switched laser assembly emits sub-nanosecond pulses at a laser power of tens and hundreds of MW. The passively Q-switched laser assembly does not require switching electronics, thereby reducing the size and complexity of the total system and improving the power efficiency. In addition, there is no need for interferometric control of the cavity dimensions, simplifying production of the device and greatly relaxing the tolerances on temperature control during its use. The result is a potentially less expensive, smaller, more robust, and more reliable Q-switched laser system with performance comparable with that of the coupled cavity Q-switched laser. The compact short cavity passively Q-switched laser assembly may be used for a large range of applications, including but not limited to high-precision ranging, robotic vision, automated production, efficient non-linear frequency conversation including harmonic generation (second harmonic, third harmonic, fourth harmonic, sum frequency generation, OPO, etc.), environmental monitoring, micromachining, spectroscopy, cosmetics and microsurgery, skin treatment, ionization spectroscopy, automobile engine ignition, and supercontinuum generation where the high peak power is required.

The fractional handpiece may be adapted to be applied to the skin of a patient and slide over the skin. In some examples, the fractional handpiece may hover over the skin of the patient and moved at a generally equidistant distance from the surface of the skin. The beam-splitting assembly may be operable to generate an array of laser beams across a segment of skin and/or to scan a laser beam emitted by the passively Q-switched laser assembly across a segment of skin. The beam-splitting assembly may provide a one-dimensional (1-D) or a two-dimensional (2-D) treated skin area coverage via a diffractive or refractive beam splitter or scanning mirror(s). For example, the beam-splitting assembly may generate a fractionated microdot line beam pattern. In some examples, the passively Q-Switched laser handpiece may contain a second or higher order harmonic generator to generate an additional laser wavelength. In some examples, the beam-splitting assembly can be a beam scanning assembly.

### Passively Q-Switched Laser Assembly

Passively Q-switched microcavity lasers with cavity lengths of about 10 mm or shorter have been investigated extensively for several decades. However, most studies reported generation of less than a few millijoule pulse energy and less than 10 MW peak power. In particular, some of the lasers were only capable to produce nanosecond laser pulse duration. Most recently, it was demonstrated the generation of 12 mJ from a Yb:YAG/Cr:YAG microchip laser. However, only ˜3.7 MW peak power was achievable due to longer pulse duration (1.8 ns). Furthermore, the laser had to be operated under cryogenic condition (e.g., 77 degrees K) which makes practical application problematic.

Single pass pumped passively Q-switched lasers have several limitations. In order to ensure the sufficient absorption of pumping energy in the laser material, the laser medium has to be sufficiently long, however longer laser medium will lead to longer emitted pulse duration. In addition, at some particular pump wavelengths, the unabsorbed pump laser can result in unwanted bleaching of saturable absorber causing failure of Q-switching operation. To overcome these above-mentioned issues, the present disclosure introduces a passively Q-switched laser assembly with double pass pumping. Double pass pumping also facilitates use of the laser medium produced from crystals which are difficult to be doped (e.g., Nd:YAG) or have a weak absorption of laser medium at the available pump laser wavelength. The double pass pumping can be made possible by applying highly reflective dielectric coating on either the output end of laser material or input end passive Q-switch for the cavity configuration where two materials (e.g., laser material and saturable absorber) are separated with a small gap. In case of monolithic configuration, the highly reflective coating is sandwiched in between laser material and saturable absorber, while two materials are bonded together. The double pass pumped short cavity laser supports efficient pump laser absorption and shorter medium length leading to shorter pulse duration as well as a more compact laser layout.

The present disclosure describes a short cavity passively Q-switched laser assembly for producing a sub-nanosecond laser pulse with high peak power exceeding 100 MW (e.g., high-powered sub-nanosecond pulsed laser beam). The operation of the laser is based on passively Q-switching, in which a passive component acts as a Q-switcher for sake of compact and low-cost design.

The passively Q-switched laser assembly with double pass pumping offers advantage over that with single pass pumping by generating much shorter pulses due to the shorter laser material used. This is because the Q-switched pulse duration is roughly proportional to the cavity length. Furthermore, for a crystal with low doping concentration or low absorption at pumping laser wavelength, double pass pumping makes it possible to obtain sufficient pump laser absorption while maintaining shorter crystal length leading to a more compact laser design. The passively Q-switched laser assembly may reduce the cavity length since there is no need to introduce bulky active component(s). In some examples, the passively Q-switched laser assembly may use a highly doped laser material and/or a saturable absorber, resulting in shorter material lengths.

The passively Q-switched laser assembly may include two functional groups: pump lenses and a laser cavity. Pump lenses are operable to direct the pump laser into the laser crystal of the laser cavity with certain spot size. The choice of pumping spot is under the tradeoff between available pumping energy and large spot size. The larger spot size leads to higher energy while requiring higher pumping energy to enable Q-switching. The laser cavity enables passively Q-switching to generate sub-nanosecond laser pulses. The laser cavity may be a monolithic cavity or a cavity with external cavity mirror(s).

### Monolithic Cavity

In a monolithic cavity, the laser medium and saturable absorber are sandwiched with a highly reflective dielectric coating at pumping wavelength and bonded with optical contact by intermolecular forces. The highly reflective dielectric coating facilitates double-pass pumping, preventing unwanted bleaching of a passive Q-switch by unabsorbed pump laser energy. Additionally, it aids in reducing the length of the laser material, supporting the generation of short pulse durations while maintaining sufficient absorption of pump energy.

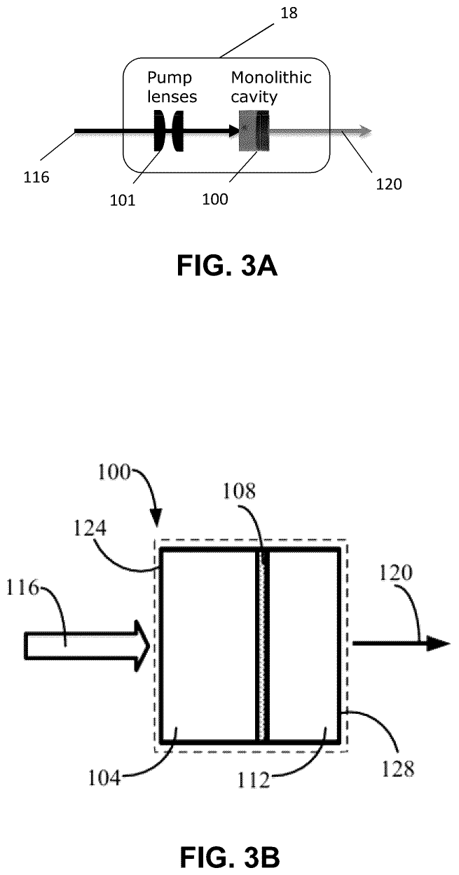

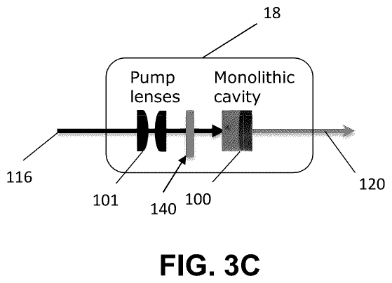

A passively Q-switched laser assembly with a monolithic cavity 100 and pump lenses 101 is shown in FIG. 3A. FIG. 3B shows the monolithic cavity 100 may include a laser medium 104, a highly reflective dielectric coating 108 for pumping laser wavelength sandwiched in between laser medium 104 and a saturable absorber 112. FIG. 3C shows the passively Q-switched laser assembly 18 may include a homogenizer 140 between the pump lenses 101 and the monolithic cavity 100. FIGS. 3A, 3B, and 3C also show a pump laser beam 116 and an output beam 120. The pump laser beam 116 may be, for example a beam with a wavelength of about 755 nm to pump a monolithic microchip laser including Nd:YAG as laser medium and Cr^{4+:}YAG as saturable absorber. Highly reflective dielectric coating 108 (highly reflecting at pump laser wavelength, about 755 nm and highly transmitting Q-switched laser wavelength, 1064 nm)) supports achieving double passing pumping and avoids unwanted bleaching of passive Q-switch 112 by unabsorbed pump laser leaking through it. Other pumping wavelengths may be used, including but not limited to diode lasers operating at 795-820 nm, or other types of solid-state emitting laser at about 795-820 nm (e.g., Ti:Sapphire).

At the input end 124 of the monolithic cavity 100, the surface of laser material 104 may be coated with a highly reflective at the laser wavelength (e.g., 1064 nm dielectric coating) and highly transmissive at pump wavelength. At the output end 128 of the monolithic cavity 100, the surface of passive Q-switch 112 may be deposited with dielectric coating partially reflective at the monolithic cavity 100 output beam wavelength. The coating 108 considers the refractive indices of laser medium and saturable absorber such that the coating functions as required when the monolithic material is formed. These two ends (124 and 128) may be arranged to be parallel and coated with dielectric coating, allowing laser oscillation occurs. The two ends may be flat surfaces or curved surfaces with curvatures operable to achieve better mode selectivity.

Diffusion bonding is commonly used to bond laser material and passive Q-switching element (e.g., a saturable absorber) to form passively Q-switched microchip laser. This method is typically accomplished at an elevated pressure and temperature, approximately 50-70% of the absolute melting temperature of the placed in contact materials. Such fabrication process involves elevated temperature and makes it difficult to deposit any form of dielectric coating in between two elements (e.g., laser medium and passive Q-switcher), in particular, highly reflective coating at pump laser wavelength. Therefore, single pass pumping can be only applied.

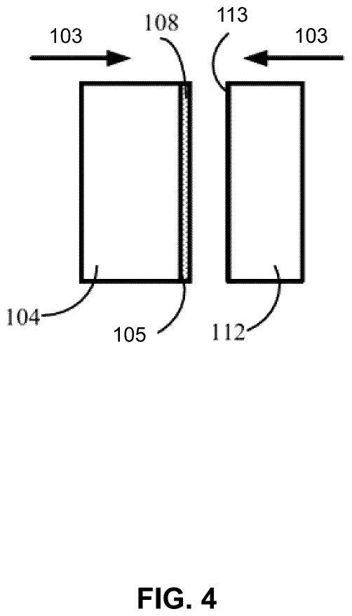

In the current disclosure, the bonding between laser medium 104 and saturable absorber 112 may be implemented as illustrated by arrows 103 through optical contact by intermolecular forces, such as Van der Waals forces, hydrogen bonds, and dipole-dipole interactions, as shown in FIG. 4. No elevated temperature and pressure are needed so that integrity of reflective dielectric coating 108 is protected.

The two surfaces of being contacted for example, 105 of laser medium 104 and 113 of saturable absorber 112 are processed in optical quality to achieve stable optical contact. The highly reflective dielectric coating at an interface between laser medium 104 and saturable absorber 112 at pump wavelength supports achieving double passing pumping and avoids unwanted bleaching of passive Q-switch by unabsorbed pump laser. Generally, the surface quality may be better than 20-10 scratch-dig. The flatness and roughness may be at least λ/4 10 A rms or better, respectively.

Laser medium 104 may be an Nd³⁺ doped material. The host material may be YAG, YAP, YLF crystals or ceramic. Non-limiting examples of the laser medium include crystals (e.g., Nd:YAG, Nd:YAP, Nd:YLF), or Nd:YAG ceramic. Saturable absorber 112 may be Chromium (Cr⁴⁺) doped crystals (e.g., YAG) or ceramic YAG. The materials for the laser medium and saturable absorber may be of the same host material or of different materials. In some examples, the laser medium and saturable absorber may be the same host material (e.g., crystal or ceramic) or monolithic composited ceramic and crystal. This is quite different from the existing microchip lasers bonded through diffusion methods where the material physical properties (e.g., melting point, thermal expansion coefficient, etc.) for the two components should be similar.

The high-energy/high peak power ultrashort pulse microchip laser facilitates efficient non-linear frequency conversation including harmonic generation (second harmonic, third harmonic, fourth harmonic, sum frequency generation, OPO, etc.) and supercontinuum generation where the high peak power is required. In contrast to the existing low-energy microchip laser, the high-energy microchip laser can provide higher energy/power at frequency converted wavelengths therefore significantly increasing measurement precision by improving signal-to-noise ratio. Most importantly, the optical arrangement is very compact and simple and supports mounting of the microchip laser in constrained space for example, in a handpiece.

### Cavity with External Mirrors

In a cavity with external mirrors, the laser cavity is configured to be a linear cavity with a cavity length shorter than 10 mm for achieving compactness and short pulse generation. In some examples, the cavity length may be less than 10 mm, less than 8 mm, or less than 5 mm. The laser cavity is intended to generate a sub-nanosecond pulsed laser beam. The sub-nanosecond laser pulse may be less than 1000 ps. In various examples, the sub-nanosecond laser pulse may range from 150 ps to less than 1000 ps, about 200 ps to about 400 ps, about 300 ps to about 500 ps, about 400 ps to about 600 ps, or about 500 ps to about 1000 ps. The sub-nanosecond laser may have a wavelength of about 1 μm (e.g., 1064 nm for Nd:YAG, 1080 nm for Nd:YAP, 1047/1053 nm for Nd:YLF).

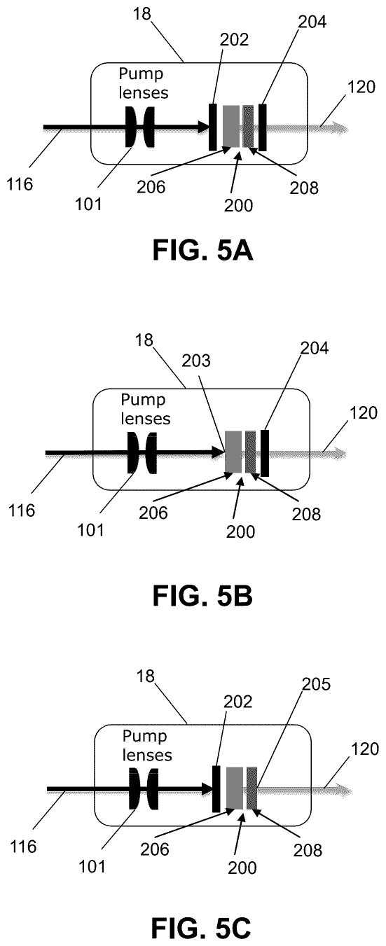

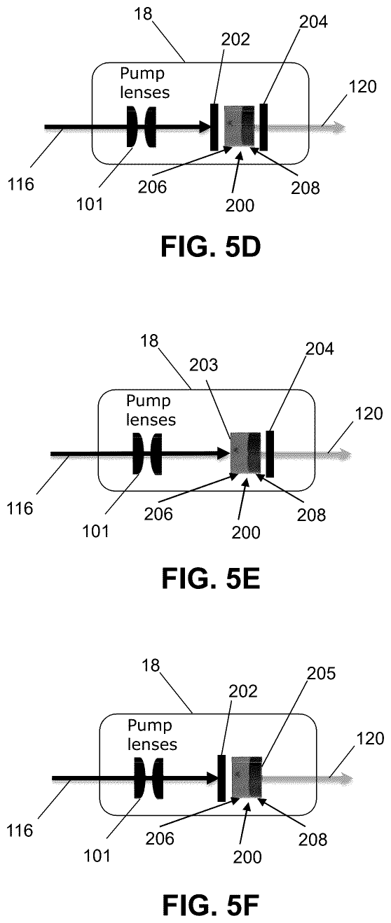

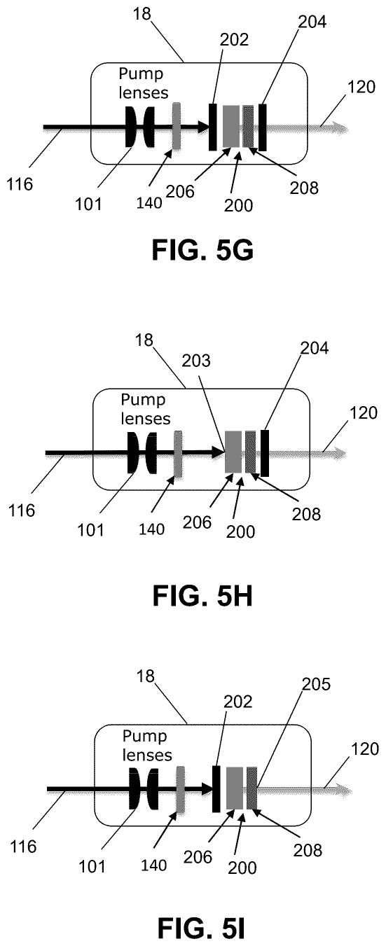

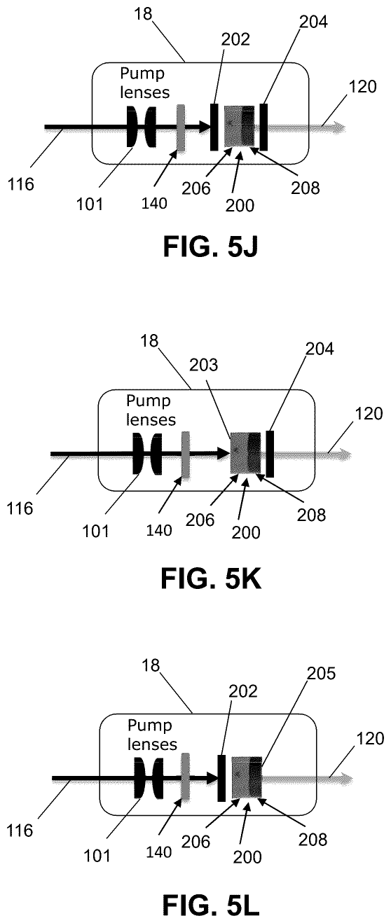

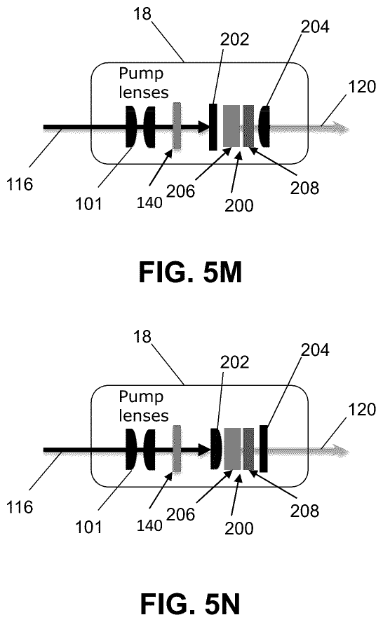

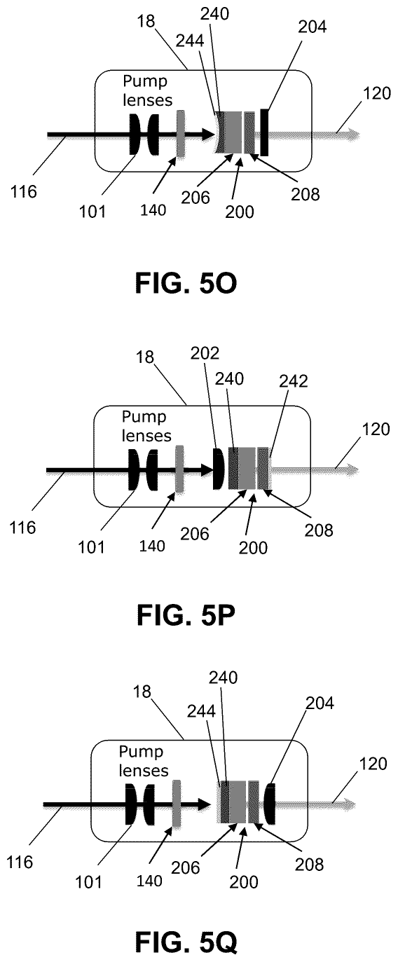

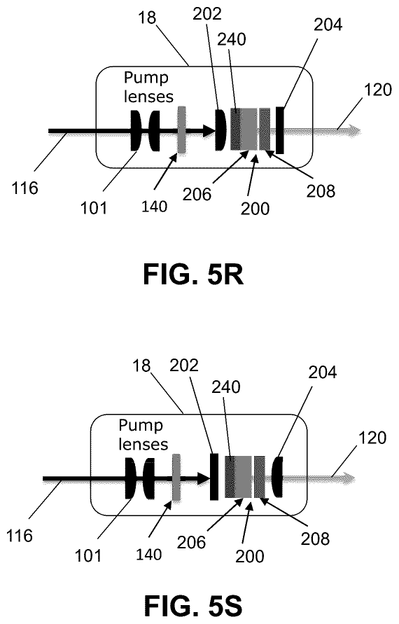

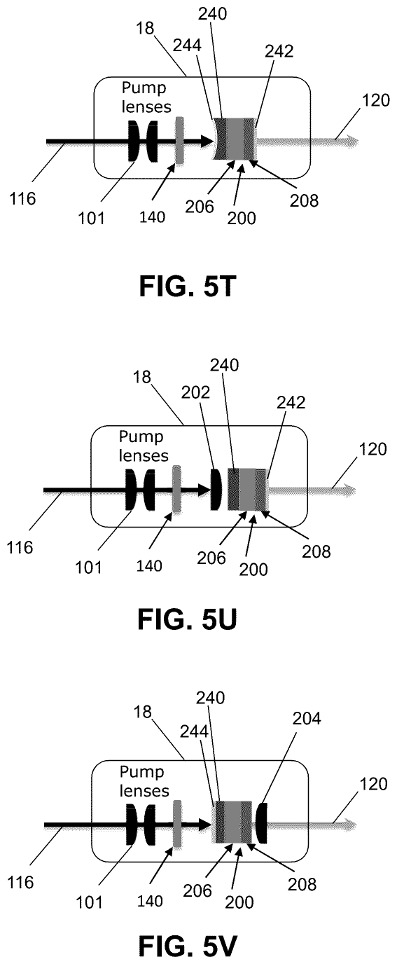

A passively Q-switched laser assembly 18 with at least one external mirror laser cavity 200 and pump lenses 101 is shown in FIGS. 5A-5L. The external mirror laser cavity may include one or more external cavity mirrors. In at least one example, the laser cavity may include two external cavity mirrors. In another example, the passively Q-switched laser assembly 18 may include at least one homogenizer placed in the pump beam path, as seen in FIGS. 5G-5L.

In particular, FIG. 5A shows the external mirror cavity 200 may include a pair of cavity mirrors forming a resonator (e.g., high reflector (HR) 202 and output coupler (OC) 204), a gain medium 206, and a saturable absorber 208 acting as a passive Q-switcher. Also shown in FIGS. 5A-5F is a pump laser beam 116 and an output beam 120. The pump laser beam 116 may be, for example a beam with a wavelength of about 755 nm. Other pumping wavelengths may be used, including but not limited to diode lasers operating at 795-820 nm, or other solid-state emitting lasers at 795-820 nm (e.g., Ti:Sapphire).

In some examples, the diode laser can have a pumping wavelength (e.g., first wavelength) of about 700 nm to about 750 nm, about 750 nm to about 800 nm, about 800 nm to about 850 nm, about 850 nm to about 900 nm, about 900 nm to about 950 nm, about 950 nm to about 1000 nm, or any wavelength therebetween. In some examples, the diode laser can have a pumping wavelength (e.g., first wavelength) of about 750 nm to about 980 nm. In some examples, the diode laser can have a pumping wavelength (e.g., first wavelength) of about 700 nm to about 725 nm, about 725 nm to about 750 nm, about 750 nm to about 775 nm, about 775 nm to about 800 nm, about 800 nm to about 825 nm, about 825 nm to about 850 nm, about 850 to about 875 nm, about 875 nm to about 900 nm, about 900 nm to about 925 nm, about 925 nm to about 950 nm, about 950 nm to about 975 nm, about 975 nm to about 1000 nm, or any wavelength therebetween.

In some other examples, one of the cavity mirrors (e.g., high reflector 202 or output coupler 204) may be replaced by depositing appropriate optical coatings on one of the end surfaces of laser gain medium 206 or saturable absorber 208 (see FIGS. 5B-5C and 5E-5F). The use of only one external cavity mirror may help reduce the cavity length, leading to shorter pulse generation. In one example, a high reflecting coating 203 may be deposited onto the input end of the laser gain medium 206 to act as high reflector while leaving output coupler 204 as one external mirror (FIG. 5B). In another example, only an external high reflector 202 may be included while a partially reflective coating 205 may be deposited onto the output end of the saturable absorber to perform the function of an output coupler (FIG. 5C).

In some examples, laser gain medium 206 may be bonded with saturable absorber 208 as one physical element for shortening cavity length, leading to shorter pulse duration, as seen in FIGS. 5D-5F. Different from the typical monolithic cavity, this monolithic element is coated with AR coatings at laser wavelength on at least one end. In addition, the coatings on the input end of this monolithic crystal may be highly transmissive at pump laser wavelength. Similar to the typical monolithic cavity, the laser medium and the saturable absorber are sandwiched with coatings which are highly reflective at the pump wavelength (e.g., first wavelength) and highly transmitting at the laser wavelength (e.g., second wavelength). In various examples, the high reflective coating 203 may be deposited onto the input end of the laser gain medium 206, acting as high reflector while a separate mirror with partially reflective coating acting as an output coupler 204 (FIG. 5E). In other examples, the saturable absorber 208 output end may be coated with a partially reflective coating acting as output coupler 205 while a separate HR mirror 202 may be present for optimizing cavity alignment (FIG. 5F). All these configurations may help in reducing cavity length, which may support shorter pulse generation and simplify the design.

In some examples, the passively Q-switched laser assembly 18 may include at least one homogenizer 140. FIGS. 5G-5L show the laser assemblies of FIGS. 5A-5F, respectively, with a homogenizer 140 placed in the pump beam path. For example, the homogenizer 140 may be located after the pump lenses 101, prior to the beam entering the passively Q-switched resonator formed by the high reflector 202, the output coupler (OC) 204), the gain medium 206, and/or the saturable absorber 208.

Instead of using a wavelength tuning element in the cavity, the wavelength selectivity may be implemented with high damage threshold optical surface coatings directly deposited on the end surfaces of the cavity mirrors with specific spectral requirements. The high reflector (HR) cavity mirror 202 may be coated to be highly transmitting at pump laser wavelength and highly reflective at laser wavelength (R≥99%) (e.g., 1064 nm for Nd:YAG). The output coupler (OC) cavity mirror may be coated with a partially reflective coating at laser wavelength.

The laser gain medium 206 may include one or more crystals. In some examples, the laser gain medium may be a laser crystal or a ceramic material. Non-limiting examples of crystals are Nd:YAG (neodymium-doped yttrium aluminum garnet), Nd:YAP (Neodymium doped yttrium aluminum perovskite), or Nd:YLF (neodymium-doped yttrium lithium fluoride). In at least one example, the laser gain medium 206 may be rare-earth ion-doped ceramic material, such as ceramic Nd:YAG. The front surface of the laser gain medium 206 may be coated with an anti-reflective coating. The back surface of the laser gain medium 206 may be coated with a highly reflective dielectric coating at pump laser wavelength to support achieving double passing pumping and to avoid unwanted bleaching of passive Q-switch by unabsorbed pump laser. Double pass pumping geometry supports sufficient pump laser absorption and shorter medium length leading to a more compact laser layout and shorter pulse duration.

The saturable absorber 208 may act as a passive Q-switcher to implement Q-switching to generate sub-nanosecond laser pulses near 1 μm. Non-limiting examples of the saturable absorber are a Cr⁴⁺:YAG crystal, a ceramic Cr⁴⁺:YAG, GaAs, or a semiconductor saturable absorber.

In some other examples, the passively Q-switched laser assembly 18 may have an unstable cavity operation, with the pair of cavity mirrors forming a resonator that is operated in unstable regime. For example, the pair of cavity mirrors can be the high reflector (HR) 202 and the output coupler (OC) 204. The unstable cavity operation may facilitate improved beam mode stability, both in mode volume and in transverse mode. Better beam mode stability may lead to improved beam quality and energy extraction increasing the brightness of the laser system. Additionally, better beam mode stability from the unstable cavity operation may facilitate improved stability of pulse duration of the pulsed laser beam at the second wavelength. The gain medium 206 may be a rare earth ion-doped gain material, e.g., Nd:YAG crystal or ceramic.

As shown in FIG. 5M, the unstable resonator may be formed by a plano high reflector 202 and a convex output coupler 204. Thus, the passively Q-switched laser assembly 18 may include the plano high reflector 202 at a proximal end, the convex output coupler 204 at a distal end, the gain medium 206, and the saturable absorber 208. As shown in FIG. 5N, the unstable resonator may be formed by a convex high reflector 202 and a plano output coupler 204. Thus, the passively Q-switched laser assembly 18 may include the convex high reflector 202 at a proximal end, the plano output coupler 204 at a distal end, the gain medium 206, and the saturable absorber 208.

Referring to FIGS. 5O-5V, the unstable resonator may include a bonded element 240. In some examples, the unstable resonator may include a bonded element 240 bonded with a proximal end of the gain medium 206. The bonded element 240 may be transparent to both the first and second wavelengths. In some examples, the bonded element 240 may be the same material as the host material of the gain medium 206, e.g. the bonded element may be YAG, while the gain medium is Nd:YAG, the same material being YAG. In other examples, the bonded element 240 may be a different material than the gain medium 206, e.g., sapphire versus Nd:YAG, sapphire and YAG being different materials. The bonding of the bonded element 240 to the gain medium 206 may be implemented via optical contact or diffusion bonding. The bonded element 240 may facilitate improved heat removal efficiency from the gain medium 206 where undesirable heat is generated in the gain medium through the adsorption of the pump energy and can dissipate more rapidly via conduction into the undoped bonded element. Additionally, the bonded element 240 may further facilitate mitigation of thermal effects in the laser, such as beam characteristic variation with the repetition rate, limited energy scaling due to thermal lensing, over stressing the gain medium leading to its fracture, etc., particularly when the laser is operating at a high repetition rate.

As shown in FIG. 5O, the unstable resonator may be formed by a coating 244 on a concave surface of the bonded element 240 and a plano output coupler 204. Thus, the passively Q-switched laser assembly 18 may include the bonded element 240, with the coating 244 on the concave surface being at a proximal end, the plano output coupler 204 at a distal end, the gain medium 206, and the saturable absorber 208. The coating 244 may highly reflect the laser at the second wavelength and highly transmit the laser at the first wavelength, thereby acting as a high reflector. As shown in FIG. 5P, the unstable resonator may be formed by a convex high reflector 202 and a coating 242 on a distal end of the saturable absorber 208. The coating 242 may be a partial reflective coating at the second wavelength. Additionally, the bonded element 240 may be an undoped material, which is transparent at both the first and second wavelengths. Thus, the passively Q-switched laser assembly 18 may include the convex high reflector 202 at a proximal end, the saturable absorber 208 at a distal end, with the coating 242 being at the distal end, and the gain medium 206. The convex high reflector 202 may highly reflect the laser at the second wavelength and highly transmit the laser at the first wavelength.

As shown in FIG. 5Q, the unstable resonator may be formed by the coating 244 on a proximal end of the bonded element 240 and a convex output coupler 204. The coating 244 may be a highly reflective coating at the second wavelength and a highly transmitting coating at the first wavelength, thereby acting as a high reflector. Additionally, the bonded element 240 may be an undoped material, which is transparent at both the first and second wavelengths. Thus, the passively Q-switched laser assembly 18 may include the bonded element 240 at a proximal end, with the coating 244 being at the proximal end, the convex output coupler at a distal end, the gain medium 206, and the saturable absorber 208.

As shown in FIG. 5R, the unstable resonator may be formed by a convex high reflector 202 and a plano output coupler 204. Thus, the passively Q-switched laser assembly 18 may include the convex high reflector 202 at a proximal end, the plano output coupler 204 at a distal end, the bonded element 240, the gain medium 206, and the saturable absorber 208. The convex high reflector 202 is designed to highly reflect the laser at the second wavelength while highly transmitting it at the first wavelength. As shown in FIG. 5S, the unstable resonator may be formed by a plano high reflector 202 and a convex output coupler 204. Thus, the passively Q-switched laser assembly 18 may include the plano high reflector 202 at a proximal end, the convex output coupler 204 at a distal end, the bonded element 240, the gain medium 206, and the saturable absorber 208. The plano high reflector 202 may highly reflect the laser at the second wavelength while highly transmitting the laser at the first wavelength.

In some examples, the unstable resonator may include a monolithic element formed by the bonded element 240 bonded with the proximal end of the gain medium 206 and the saturable absorber 208 bonded with a distal end of the gain medium 206. The bonded element 240 may be transparent to both the first and second wavelengths. In some examples, the bonded element 240 may be the same material as the host material of the gain medium 206. In other examples, the bonded element 240 may be a different material than the gain medium 206. The bonding of the bonded element 240 and/or the saturable absorber 208 to the gain medium 206 may be implemented via optical contact or diffusion bonding.

As shown in FIG. 5T, the unstable resonator may be formed by the coating 244 on the proximal end of the concave bonded element 240 and the coating 242 on the distal end of the saturable absorber 208. The coating 244 may be a highly reflective coating at the second wavelength and a highly transmitting coating at the first wavelength, thereby acting as a high reflector. Additionally, the bonded element 240 may be an undoped transparent material. The coating 242 may be a partial reflecting coating at the second wavelength. Thus, the passively Q-switched laser assembly 18 may include the bonded element 240 at a proximal end, with the coating 244 being at the proximal end, the saturable absorber 208 at a distal end, with the coating 242 being at the distal end, and the gain medium 206 bonded to both the bonded element 240 and the saturable absorber 208. Parallelism between the proximal end of the bonded element 240 and the distal end of the saturable absorber 208 may be within 10 arc second.

As shown in FIG. 5U, the unstable resonator may be formed by a convex high reflector 202 and the coating 242 on the distal end of the saturable absorber 208. The coating 242 may be a partial reflecting coating at the second wavelength. The convex high reflector 202 may highly reflect at the second wavelength while highly transmitting at the first wavelength, thereby acting as a high reflector. Thus, the passively Q-switched laser assembly 18 may include the convex high reflector 202 at a proximal end, the saturable absorber 208 at a distal end, with the coating 242 being at the distal end, and the gain medium 206 bonded to both the bonded element 240 and the saturable absorber 208.

As shown in FIG. 5V, the unstable resonator may be formed by a convex output coupler 204 and the coating 244 on the proximal end of the bonded element 240. The coating 244 may be a highly reflective coating at the second wavelength and a highly transmitting coating at the first wavelength, thereby acting as a high reflector. The bonded element 240 may be an undoped transparent material at both the first and second wavelengths. Thus, the passively Q-switched laser assembly 18 may include the bonded element 240 at a proximal end, with the coating 244 being at the proximal end, the convex output coupler 204 at a distal end, and the gain medium 206 bonded to both the bonded element 240 and the saturable absorber 208.

### Second Harmonic Generation Assembly

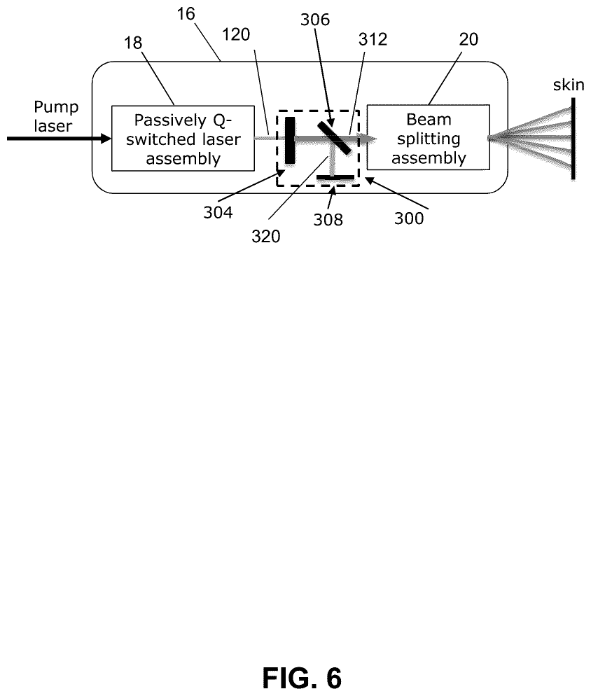

FIG. 6 is an example of a fractional handpiece 16 with a second harmonic generation assembly 300 between the passively Q-switched laser assembly 18 and the beam-splitting assembly 20. The laser wavelength out of the passively Q-switched cavity may be converted to other wavelengths through non-linear frequency generation. In some examples, the second harmonic generation assembly 300 may include a frequency doubling crystal 304, a dichroic mirror 306, and a beam dump 308. In an example, the frequency doubling crystal may generate a second harmonic wavelength in the visible wavelength for enhancing melanin absorption.

In some examples, the frequency doubling crystal 304 may be a second harmonic generation (SHG) crystal. Non-limiting examples of frequency doubling crystals include lithium niobate (LiNbO₃), potassium titanyl phosphate (KTP=KTiOPO₄), lithium triborate (LBO=LiB₃O₅), or any other SHG crystals. For generation of a stable linearly polarized Q-switched laser, specially cut Nd:YAG (e.g. [100], or/and Cr⁴⁺:YAG (e.g., [110] cut) may be used.

The dichroic mirror may be operable for transmitting the second harmonic laser while rejecting the residual fundamental wavelength. For example, the frequency doubling crystal 304 receives output beam 120 from the passively Q-switched laser assembly and transforms it into two beams—one beam 320 maintaining the original wavelength (frequency) of the output beam 120 and a beam 312 having a frequency two times higher than the output beam 120. In at least one example, the output beam 120 has a wavelength of 1064 nm, and the beam with the doubled frequency has a wavelength of 532 nm. The dichroic mirror 306 may be a beam splitter that splits and directs beams 320 and 312 in different directions. The beam maintaining the original wavelength 320 may be directed to the beam dump 308. In an example, the beam dump is used to block the rejected fundamental wavelength laser. The output beam of the second harmonic generation assembly 312 may then be passed into the beam-splitting assembly 20.

### Handpiece

One of the potential and promising applications for the passively Q-switched laser assembly producing sub-nanosecond laser pulses may be in cosmetic and medical laser systems. The high-energy short pulse passively Q-switched laser assembly supports the packaging of the passively Q-switched laser assembly into a handpiece to perform meaningful aesthetic treatment and in particular fractional skin rejuvenation. It has been demonstrated clinically that for laser pulses of a few hundred picoseconds with ˜mJ per laser beam are sufficient enough to cause tissue or skin micro-injury through laser induced optical breakdown (LIOB) or melanin assistant optical breakdown. The subsequent collagen remodeling stimulated by such micro-injury will result in skin rejuvenation. The current passively Q-switched laser assembly is capable of generating more than 40 mJ, ˜300 ps laser pulses with wavelength of 1064 nm. Therefore, the output energy from the passively Q-switched laser assembly may be split into a plurality of micro-beams using a beam-splitting assembly 20. For example, the beam from the passively Q-switched laser assembly may be split into at least 2 micro-beams, at least 5 micro-beams, at least 10 micro-beams, at least 15 micro-beams, or at least 30 micro beams. Other numbers of micro-beams are contemplated. Each micro-beam may have up to 5 mJ, up to 4 mJ, up to 3 mJ, up to 2 mJ, or up to 1 mJ of laser energy which is sufficient for effective skin treatment. Each of the micro-beams may be focused by a lens in the beam-splitting assembly to generate a plurality of micro-dots of a few hundred micron in diameter.

Skin treatment usually requires irradiation of a two-dimensional skin area. Fractional skin treatment may use micro-beams or fractional beams with scanning mirrors or other scanning means. There are a number of approaches to implement two-dimensional micro-beam patterns. For example, the laser beam may be split into a one-dimensional array of micro-beams and the one-dimensional array of micro-beams may be manually slid over the skin. Another approach may use a scanning system to scan the array of micro-beams in one or two directions/axes.

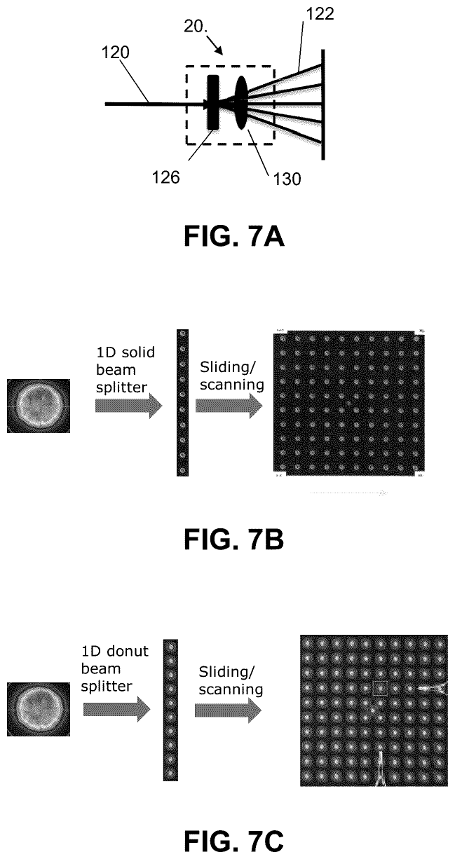

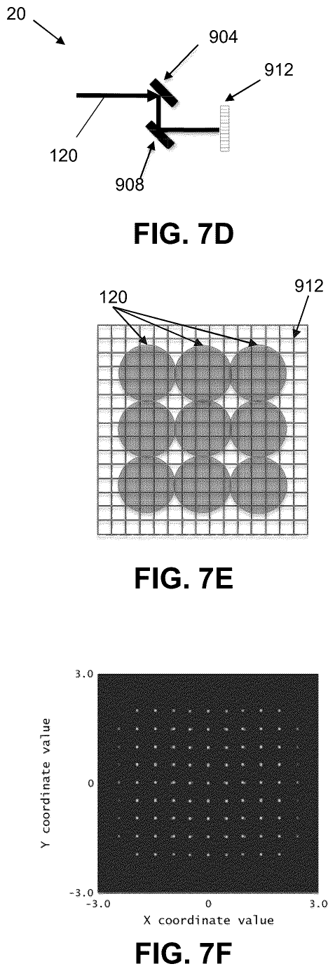

FIG. 7A shows an example 1-D beam-splitting assembly 20 with a 1-D beam splitter 126 and a focusing lens 130. The beam-splitting assembly 20 is designed to split the incoming one solid beam 120 into an array of multiple micro-beams 122 with a combination of the 1-D beam splitter 126 and the focusing lens 130. In some examples, the 1-D beam splitter may be a 1-D diffractive optical beam splitter or a scanner.

The micro-beam size in the focal plane may be in the range of about 10 μm to about 300 μm in diameter. In various examples, the micro-beam size may be up to 10 μm, up to 20 μm, up to 50 μm, up to 100 μm, up to 150 μm, up to 200 μm, up to 250 μm, or up to 300 μm in diameter. In some examples, the micro-beam may have a diameter in the focal plane ranging from about 10 μm to about 50 μm, about 25 μm to about 75 μm, about 50 μm to about 100 μm, about 75 μm to about 125 μm, about 100 μm to about 150 μm, about 125 μm to about 175 μm, about 150 μm to about 200 μm, about 200 μm to about 250 μm, or about 250 μm to about 300 μm.

The split beam may be solid micro-dot or donut shape (e.g., dot surrounded by a ring). For example, the 1-D beam-splitting assembly may include an axicon diffractive optic to form a ring or donut shaped beam, a 1-D beam splitter, and a focusing lens. FIG. 7B shows an example output of a 1-D solid beam splitter and a resulting scan of the micro-dot array. FIG. 7C shows an example output of a 1-D donut beam splitter and a resulting scan of the micro-dot array. The donut-shaped beam array helps increase surface coverage to reduce the number of passes for treatment.

In other examples, the sub-nanosecond laser generated from the passively Q-switched laser assembly may be directed to a lens array to form fractionated microbeams by a pair of scanning mirrors. FIG. 7D shows an example beam-splitting assembly 20 with a pair of scanning mirrors, e.g. galvanometer driven mirrors (Galvo mirrors) 904 and 908 that project or scan laser beam 120 onto a lens array 912. In some examples, a first scanning mirror 904 may provide in-plane rotation and a second scanning mirror 908 may provide out of plane rotation, while lens array 912 splits laser beam 120 into a plurality of fractionated micro-beams. The combination of a pair of scanning mirrors and lens array optic may generate microdot array with large surface coverage. The movement of two scanning mirrors can direct the incident beam across the lens array surface in 2-dimensions to increase surface coverage. To avoid the overlap of the scanning spots or leave spare untreated area, the scanning may be appropriately programmed so that the spots projected on the lens array after one scanning cycle are next to each other. In at least one example, passively Q-switched laser energy is high enough so that each laser beam from the sub-nanosecond laser can cover multiple lenslets to generate multiple micro-dots with sufficient energy per microdot for meaningful treatment. The scanning mirror pair may scan the laser beam to form a 2-D pattern and cover a larger area of lenslets, as shown in FIG. 7E. After the scanning system completes one cycle, a 2-D micro-dot array may be generated in the skin with larger surface coverage, as seen in FIG. 7F. The scanning of the incident sub-nanosecond laser beam may be in sequence or in random order.

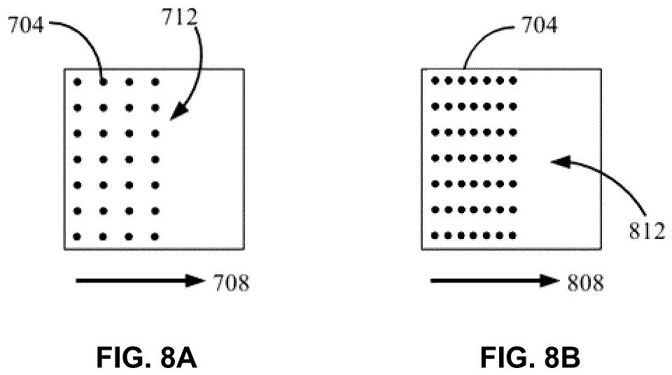

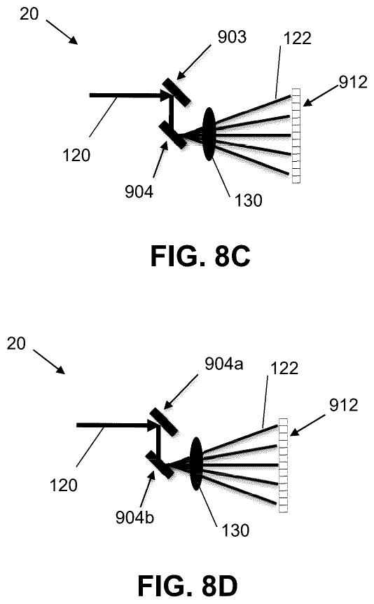

For 1-D beam splitting, the speed of scanning may affect the density or coverage of the micro-dot array across the treatment area. The generated line of multiple micro-beams may be extended to a two-dimensional micro-beam array by manually sliding the handpiece along the direction perpendicular to the line of the micro-dots guided with a positional sensor or scanning the line of microdots with a scanner. In some examples, the scanner can be an x-y scanner. In some examples, the positional sensor may include one or more rollers, one or more accelerometers, or combinations thereof. For example, as seen in FIG. 8A, manual movement of the fractional handpiece in a direction perpendicular to the 1-D fractionated micro-dot line 704 as shown by arrow 708 may generate a 2-D fractional beam pattern 712. In some examples, operation of the passively Q-switched laser may be triggered and synchronized by the roller(s), accelerometer(s), and/or x-y scanner(s). The fractional treated skin area coverage may be changed by varying the number of fractionated micro-dots 704 in a 1-D line and/or movement speed of the fractionated handpiece. The movement speed of the fractionated handpiece may determine the spacing between multiple micro-dot arrays. FIG. 8A is an example of a relatively sparse fractionated micro-dot 704 array in a 1-D line (e.g. 7 micro-dots) moved at speed 708. FIG. 8B is an example of the same density fractionated micro-dot 704 array in a 1-D line moved at speed 808. For example, movement speed 708 in FIG. 8A is faster than the same handpiece with movement speed 808 in FIG. 8B. Accordingly, a denser 2-D pattern 812 of fractionated micro-dots 704 is generated. Slower sliding may therefore result in higher coverage of the fractionated beam and faster sliding may result in lower coverage of the fractionated beam over the treatment area. In some examples, the number of fractionated micro-dots in a 1-D line may be adjusted by changing the beamsplitter. For example, the beamsplitter may be a snap-on disposable optic tip operable to connect to the handpiece. Snap-on tips may allow routine easy cleaning of the beamsplitter optics and offer the user different tip designs including different attenuation levels, and different micro-dot arrangements (more or less micro-dots per column, and different micro-dot densities). The user may select different tips based on the number of micro-dots desired per column or same number of micro-dots but with denser or less dense micro-dots.

FIG. 8C shows an example beam-splitting assembly 20 (e.g., beam scanning assembly) with a fixed mirror 903 and a scanning mirror 904 (e.g., a Galvo mirror) to project or scan laser beam 120 onto the lens 130. The beam-splitting assembly 20 also includes the focusing lens 130 and is designed to scan the incoming one solid beam 120 into an array of multiple micro-beams 122 with a combination of the scanning mirror 904 and the focusing lens 130. The fixed mirror 903 may be used to direct the laser beam to the scanning mirror 904. The scanning mirror 904 may rapidly scan the incoming laser beam to generate the array of multiple micro-beams 122. The array of multiple micro-beams 122 may generate a single line of micro-dots. The spacing between the micro-beams 122 may be adjusted by controlling the scanning angle of the scanning mirror 904. Additionally, the number of micro-beams 122 may be adjusted by controlling the programming of the speed of the scanning mirror 904. The focusing lens 130 may be used to focus each beam into a micro-dot on a treatment plane, with the micro-dot ranging from 50 um to 500 um. The beam-splitting assembly 20 may extend the generated line of multiple micro-beams to a two-dimensional micro-beam array by manually sliding the handpiece along the direction perpendicular to the line of the micro-dots guided with a positional sensor or scanning the line of microdots with a scanner. The one or more rollers and/or the scanning mirror 904 may be temporally synchronized with the laser pulsing of the passively Q-switched laser. The one or more rollers may be on a tip of the fractional handpiece, and the tip may be disposable. The treatment associated with this configuration is commonly referred to as ‘sliding mode’.

FIG. 8D shows an example beam-splitting assembly 20 with two scanning mirrors 904 (e.g., Galvo mirrors) to project or scan laser beam 120 onto the lens 130. The beam-splitting assembly 20 also includes the focusing lens 130 and is designed to split the incoming one solid beam 120 into an array of multiple micro-beams 122 with a combination of the scanning mirrors 904 and the focusing lens 130. The scanning mirrors 904 may include a first scanning mirror 904a to scan via an out of plane rotation and a second scanning mirror 904b to scan via an in-plane rotation. Thus, the two scanning mirrors 904 may scan the incoming laser beam along two perpendicular directions (e.g., X axis and Y axis) to create a two-dimensional micro-beam pattern. The coverage and/or the density of the micro-dot surface may be adjusted by controlling the scanning angle and/or the programmed speed of the scanning mirrors 904. The focusing lens 130 may be used to focus each beam into a micro-dot on a treatment plane, with the micro-dot ranging from 50 μm to 500 μm. The two scanning mirrors 904 may be temporally synchronized with the laser pulsing of the passively Q-switched laser. In this configuration, the two-dimensional microbeam pattern can be generated without the involvement of rollers, hence categorizing the treatment as ‘stamping mode’. Alternatively, in a scenario akin to the one depicted in FIG. 8C with a single scanning mirror, roller(s) may also be integrated to facilitate handpiece movement, referred to as ‘sliding mode’.

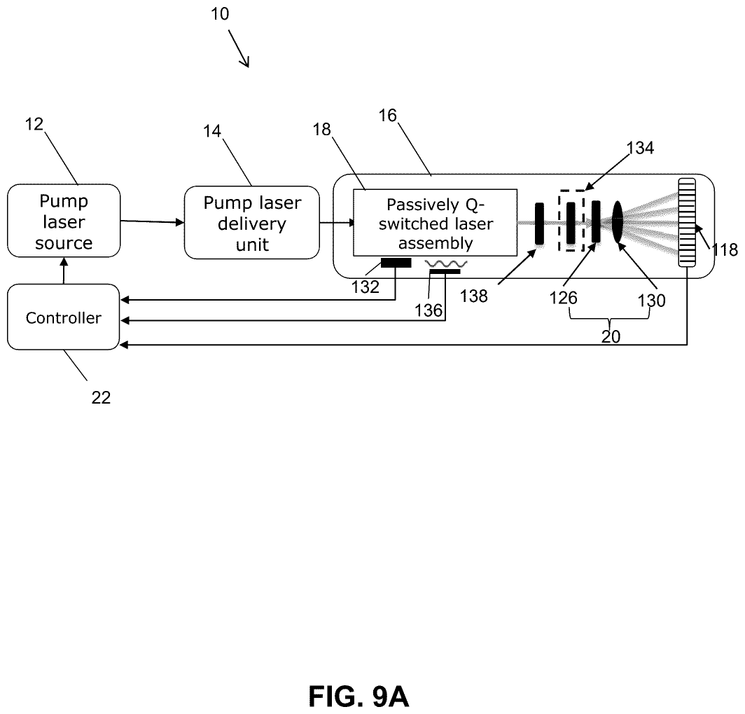

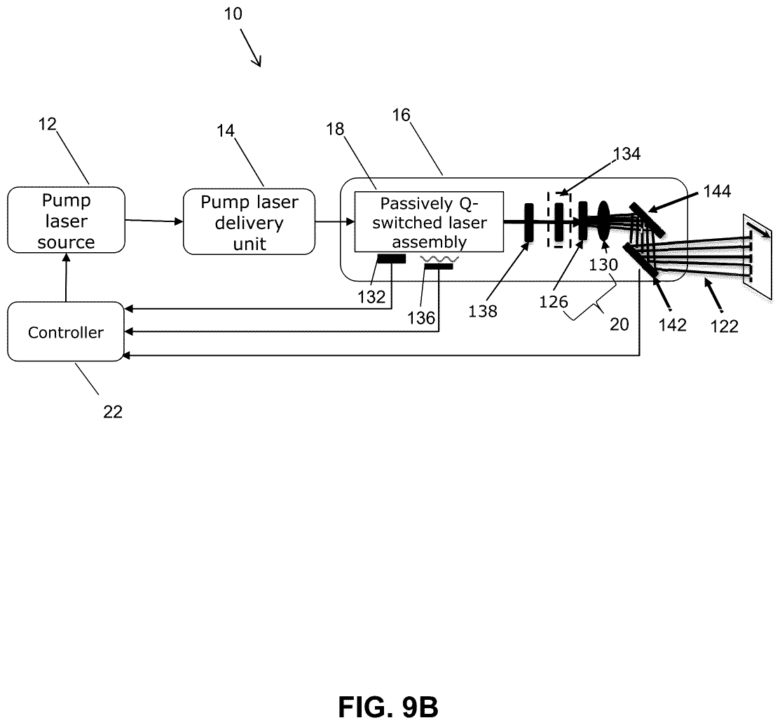

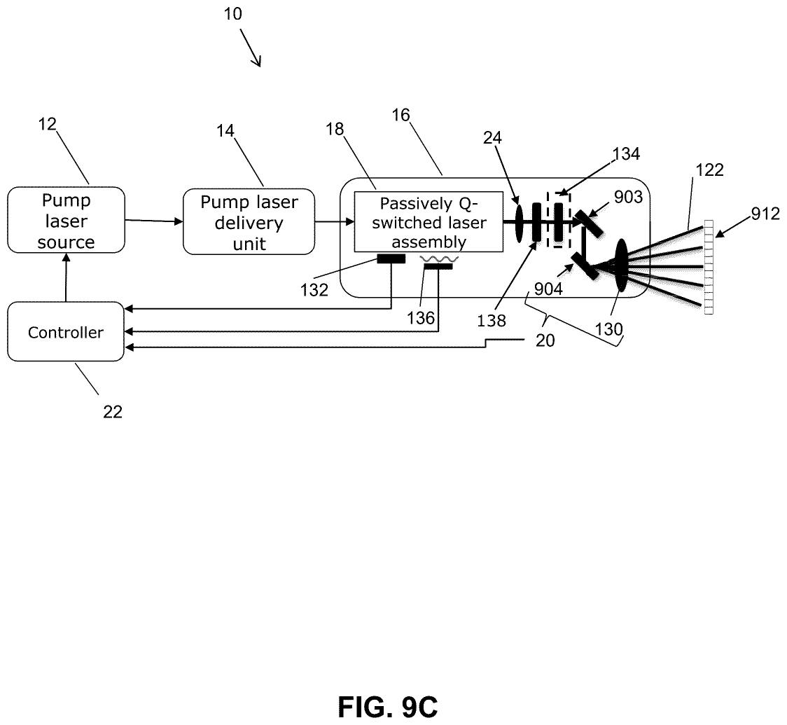

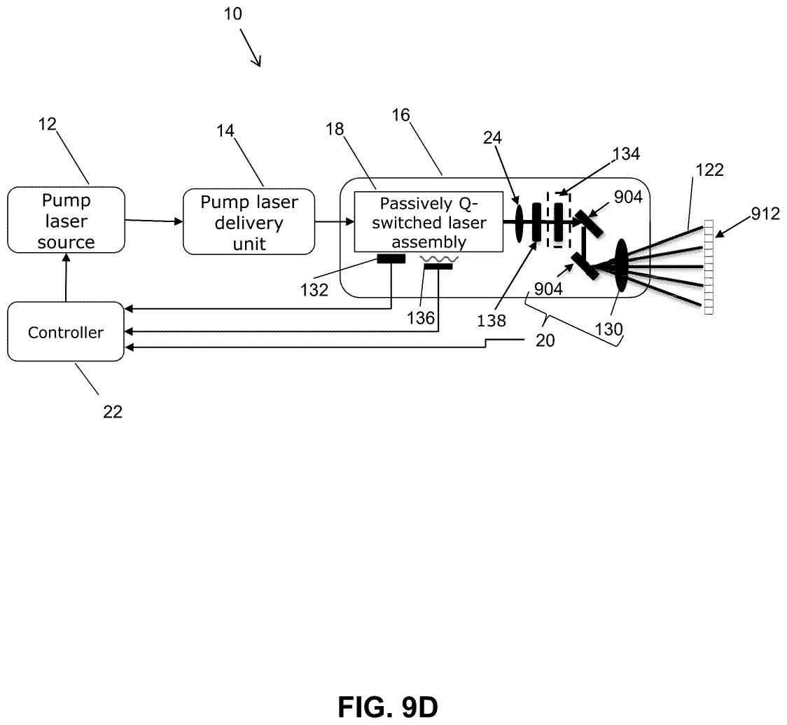

FIG. 9 shows examples of fractional handpieces including a beam splitting assembly 20 with one or two scanning mirrors or with no scanning mirror.

In one example, the fractional handpiece 16 may not involve any scanning mirror, shown in FIG. 9A. It is operable for manual movement of the handpiece. For skin treatment, the caregiver or user may manually slide the fractional handpiece with a 1-D beam splitter over the treated skin area. The fractional handpiece may physically contact the skin or may be at a distance above the surface of the skin as the user slides the handpiece over the patient's skin. In the course of the sliding movement, the passively Q-switched laser assembly may generate sub-nanosecond laser pulses forming a 1-D fractionated micro-dot line.

The fractional handpiece may include one or more positional sensors. For example, the positional sensor may include one or more rollers, one or more accelerometers, or combinations thereof. In some examples, the fractional handpiece may include one or more positional rollers for manual movement of the handpiece along a direction perpendicular to the 1-D fractionated micro-spot line to form a 2-D micro-spot pattern. The rollers and/or accelerometers may be used to determine when the pump laser should be fired again. For example, as the 1-D array is moved across the skin, the rollers may track the distance the handpiece has moved and signal to the pump laser source to fire again after the handpiece has moved a set distance. The set distance may range from about 400 μm to about 800 μm. For example, the set distance may be about 400 μm, about 500 μm, about 600 μm, about 700 μm, or about 800 μm. The rollers may also be used as a safety feature, such that when the roller stops, the pump laser source stops. This may prevent tissue damage if the movement of the handpiece is stopped or paused. Alternatively, the laser may be set to fire at a constant rate, and the rollers used to measure the speed of the handpiece movement across the skin surface. The user may receive feedback from the roller if the speed is too fast or too slow. For example, feedback may include lights, different color lights, or tactile vibrations that are initiated when improper scan speeds are triggered. The rollers may also be used to automatically trigger the pump laser and/or may be used to synchronize the laser pulses. In some examples, operation of the passively Q-switched laser may be triggered and synchronized by the roller(s). The one or two rollers may be operable for manually sliding the fractional handpiece over a target area. Manually sliding the fractional handpiece may generate a 2-D fractional beam pattern guided by one or two rollers. The fractional coverage may be changed by varying sliding speed. Slower movement leads to higher surface coverage.

The system 10 may include a pump laser source 12, a pump laser delivery unit 14, a fractional handpiece 16, and a controller 22. As described above, the fractional handpiece 16 may include a passively Q-switched laser assembly 18 and a beam-splitting assembly 20, which include a 1-D beam splitter 126 and a focusing lens 130. In one example, this 1-D splitter may be a diffractive or refractive optics, capable of dividing a single laser beam from the passively Q-switched assembly into multiple microbeams. In this configuration, the energy of each microbeam is only a fraction of the energy of the incoming laser beam.

The fractional handpiece 16 may further include a fast photodetector 132 or some other means of sensing the sub-nanosecond laser pulse. In an example, the photodetector may communicate with the controller to shut down the pump laser to avoid double or multiple pulsing. In an example, laser pulsing may be synchronized with roller rotation. In some examples, a homogenizer 138 (either diffractive based or refractive based) may be added to the fractional handpiece before the beam splitting array to correct the beam characteristic changes at different repetition rates and to homogenize the beam profile before delivering to skin. In other examples, a homogenizer 140 may be added to the fractional handpiece in the pump beam path before passively Q-switched laser assembly to mitigate pump spot size change in the laser gain medium due to the pump beam divergence changes at different repetition rates as well as to avoid hot spots in the laser gain medium due to fiber bending. In some examples, a homogenizer may be added to the fractional handpiece after the passively Q-switched laser assembly to mitigate beam characteristic variation at different repetition rates as well as deliver uniform beam profile to skin. In various embodiments, the fractional handpiece may include a first homogenizer 140, a second homogenizer 134, or combinations thereof.

The fractional handpiece may also include a vibrator 136, buzzer, mechano-oscillator, or sound system to warn an operator if there is a malfunction of the laser, such as laser misfiring. In addition, the vibrator may be used to provide tactile feedback to help a user control their scan speed by providing feedback to the user if the sliding speed is too slow or too fast. For example, the handpiece may buzz or shake if it is moving too fast. In additional examples, the fractional handpiece 16 may further include an attenuator 134 to achieve appropriate energy for the treatment of different skin types or LIOB depth. The attenuator may a neutral density filter or polarization based elements (such as polarizer cube). The photodetector 132, the vibrator 136, the positioning sensor (e.g., the roller 118), and/or the pump source laser 12 may be operatively connected to the controller 22. Controller 22 is a functional electronic system used to receive the electric signals from roller 118, vibrator 136 and photo sensor 132, process them and provide feedback signal to pump laser source for controlling pump laser on and off. The components of the fractional handpiece may be small enough to be packed into the handpiece 16. Such handpiece 16 may be operable to generate a sub-nanosecond laser beam, allowing for fractional treatment for skin rejuvenation.

FIG. 9B is an example system with a fractional handpiece with a scanning mirror. In some examples, the scanning mirror may be synchronized with laser pulsing. The system 10 may include a pump laser source 12, a pump laser delivery unit 14, a fractional handpiece 16, and a controller 22. As described above, the fractional handpiece 16 may include a passively Q-switched laser assembly 18 and a beam-splitting assembly 20, which include a 1-D beam splitter 126 and a focusing lens 130.

The beam-splitting assembly may further include a fixed mirror 144 and a rotational mirror 142 which scans a line of micro-beams 122 to form a 2-D micro-spot pattern. The fractional coverage can be adjusted by varying the scanning angle of rotational mirror 142, thereby altering the spacing between neighboring lines. In an example, the rotational mirror may be a galvo-mirror operable for out of plane rotation.

The fractional handpiece 16 may further include a fast photodetector 132 or some other means of sensing the sub-nanosecond laser pulse which shuts down the pump laser and avoids double or multiple pulsing. In an example, laser pulsing may be synchronized with rotational mirror rotation. In some examples, a homogenizer 138 (either diffractive based or refractive based) may be added to the fractional handpiece before the beam-splitting assembly to correct the beam characteristic changes at different repetition rates. In other examples, a homogenizer 140 may be added to the fractional handpiece in the pump beam path before passively Q-switched laser assembly to mitigate pump spot size change in the laser gain medium due to the pump beam divergence changes at different repetition rates as well as to avoid hot spots in the laser gain medium due to fiber bending. In various embodiments, the fractional handpiece may include a first homogenizer 140, a second homogenizer 138, or combinations thereof. The fractional handpiece may also include a vibrator 136, buzzer, mechano-oscillator, or sound system to warn an operator if there is a malfunction of the laser, such as laser missing fire. In additional examples, the fractional handpiece 16 may further include an attenuator 134 to achieve appropriate energy for the treatment of different skin types or LIOB depth. The attenuator may be a neutral density filter or polarization-based elements (such as a polarizer cube). The photodetector 132, the vibrator 136, the roller 118, and/or the pump source laser 12 may be operatively connected to the controller 22, as described above. The components of the fractional handpiece may be small enough to be packed into the handpiece 16. Such handpiece 16 may be operable to generate a picosecond laser beam, allowing for fractional treatment for skin rejuvenation.

FIG. 9C is an example system with a fractional handpiece with a scanning mirror to create 1-D microbeam array. In some examples, the scanning mirror may be synchronized with laser pulsing. The system 10 may include a pump laser source 12, a pump laser delivery unit 14, a fractional handpiece 16, and a controller 22. As described above, the fractional handpiece 16 may include a passively Q-switched laser assembly 18 and a beam-splitting assembly 20. The beam-splitting assembly 20, such as that previously described herein with respect to FIG. 8C, may include a fixed mirror 903 and a scanning mirror 904 (e.g., a Galvo mirror) to project or scan laser beam 120 onto the lens array 912 as guided by one or more rollers. The beam-splitting assembly 20 may also include a focusing lens 130 and is designed to focus each beam to form 1-D microdot array 122 with a combination of the scanning mirror 904 and the focusing lens 130. The one or two rollers may be operable for manually sliding the fractional handpiece over a target area. Manually sliding the fractional handpiece rollers along a direction perpendicular to the 1-D fractionated micro-spot line to form a 2-D micro-spot pattern. The fractional coverage may be changed by varying sliding speed and scanning mirror angle. Reduced movement speed correlates with increased surface coverage. Similarly, decreasing the scanning mirror's angle of movement also yields greater surface coverage. In some examples, operation of the passively Q-switched laser may be triggered and synchronized by the roller(s).

The fractional handpiece 16 may further include a fast photodetector 132 or some other means of sensing the sub-nanosecond laser pulse which shuts down the pump laser and avoids double or multiple pulsing. In an example, laser pulsing may be synchronized with the scanning mirror 904. In some examples, a homogenizer 138 (either diffractive based or refractive based) may be added to the fractional handpiece before the beam-splitting assembly to correct the beam characteristic changes at different repetition rates. In other examples, a homogenizer 140 may be added to the fractional handpiece in the pump beam path before passively Q-switched laser assembly to mitigate pump spot size change in the laser gain medium due to the pump beam divergence changes at different repetition rates as well as to avoid hot spots in the laser gain medium due to fiber bending. In various embodiments, the fractional handpiece may include a first homogenizer 140, a second homogenizer 138, or combinations thereof. The fractional handpiece may also include a vibrator 136, buzzer, mechano-oscillator, or sound system to warn an operator if there is a malfunction of the laser, such as laser missing fire. In additional examples, the fractional handpiece 16 may further include an attenuator 134 to achieve appropriate energy for the treatment of different skin types or LIOB depth. The attenuator may be a neutral density filter or polarization-based elements (such as polarizer cube). The photodetector 132, the vibrator 136, the roller 118, the pump source laser 12, and/or the beam-splitting assembly 20 (e.g., the scanning mirror 904) may be operatively connected to the controller 22, as described above. The components of the fractional handpiece may be small enough to be packed into the handpiece 16. Such handpiece 16 may be operable to generate a picosecond laser beam, allowing for fractional treatment for skin rejuvenation.

FIG. 9D is an example system with a fractional handpiece with two scanning mirrors. In some examples, the scanning mirrors may be synchronized with laser pulsing. The system 10 may include a pump laser source 12, a pump laser delivery unit 14, a fractional handpiece 16, and a controller 22. As described above, the fractional handpiece 16 may include a passively Q-switched laser assembly 18 and a beam-splitting assembly 20. The beam-splitting assembly 20, such as that previously described herein with respect to FIG. 8D, may include two scanning mirrors 904 (e.g., Galvo mirrors) to project or scan laser beam 120 onto the treatment plane. The beam-splitting assembly 20 may also include a focusing lens 130 and is designed to scan the incoming one solid beam 120 to form an array of multiple micro-beams 122 with a combination of the scanning mirrors 904 and the focusing lens 130.

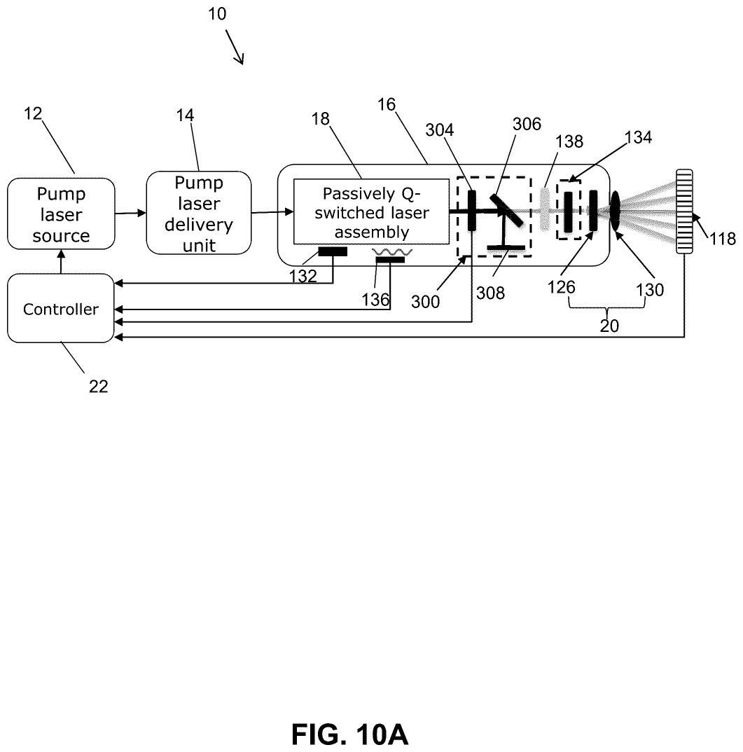

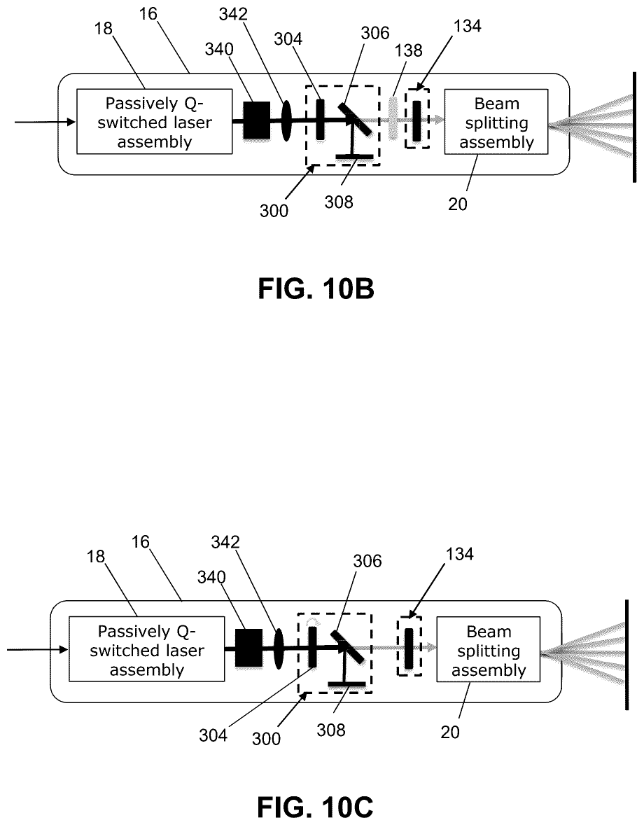

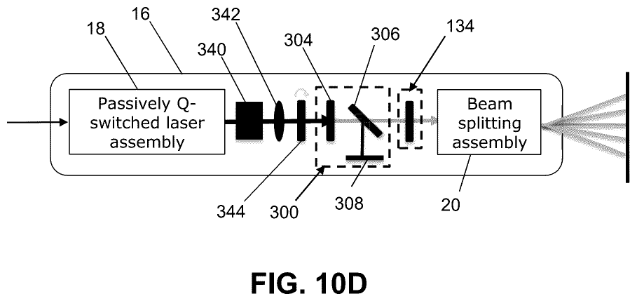

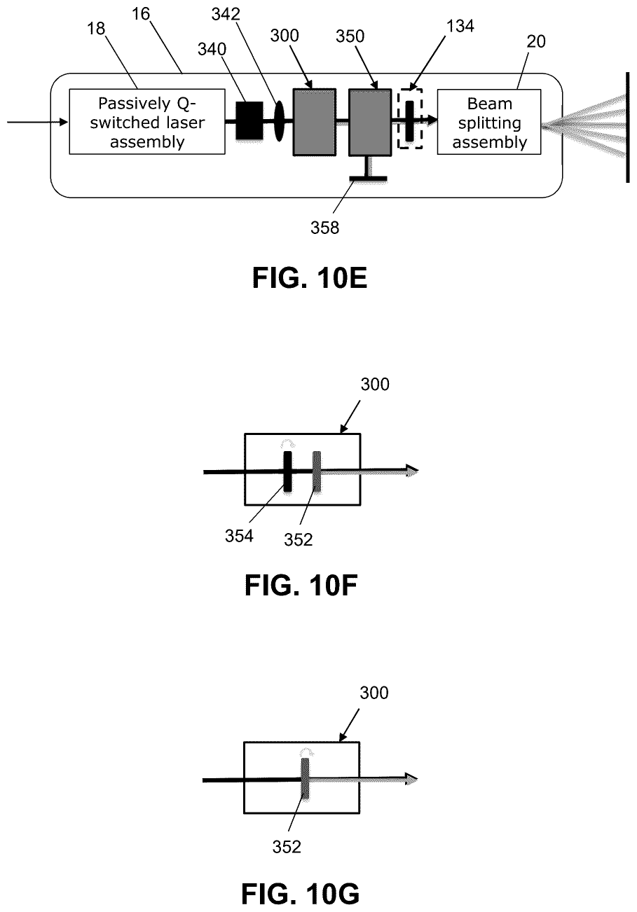

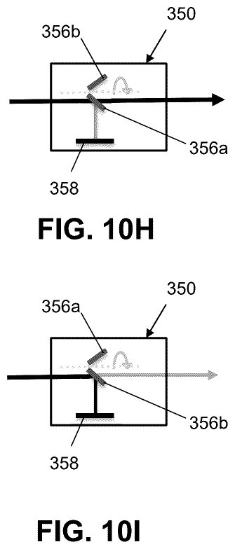

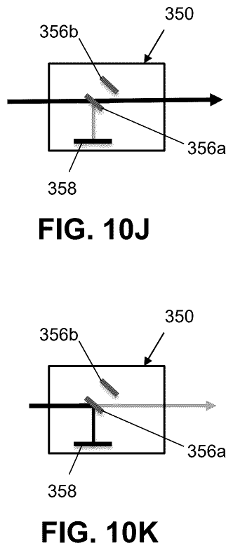

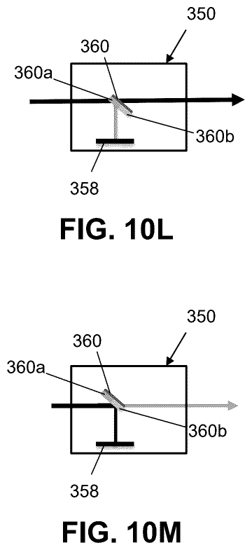

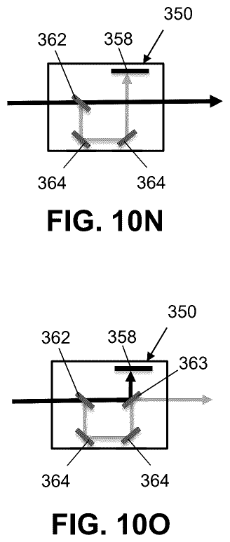

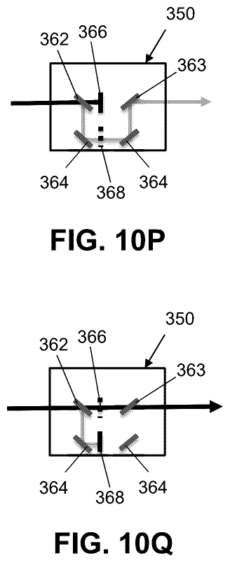

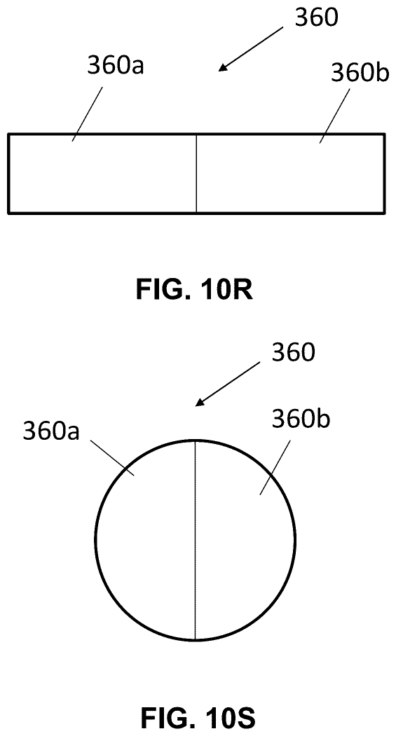

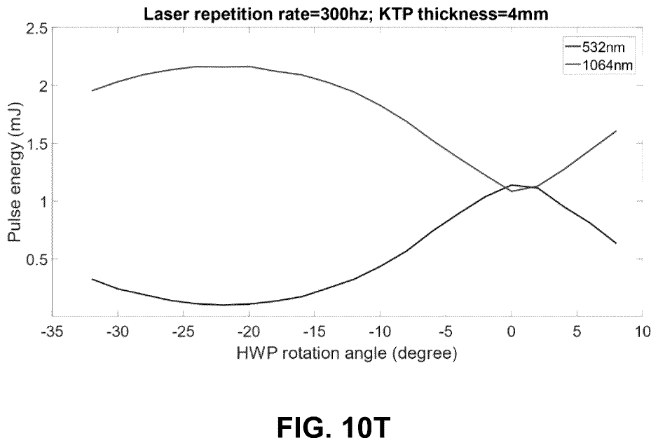

The fractional handpiece 16 may further include a fast photodetector 132 or some other means of sensing the sub-nanosecond laser pulse which shuts down the pump laser and avoids double or multiple pulsing. In an example, laser pulsing may be synchronized with the scanning mirrors 904. In some examples, a homogenizer 138 (either diffractive based or refractive based) may be added to the fractional handpiece before the beam-splitting assembly to correct the beam characteristic changes at different repetition rates. In other examples, a homogenizer 140 may be added to the fractional handpiece in the pump beam path before passively Q-switched laser assembly to mitigate pump spot size change in the laser gain medium due to the pump beam divergence changes at different repetition rates as well as to avoid hot spots in the laser gain medium due to fiber bending. In various embodiments, the fractional handpiece may include a first homogenizer 140, a second homogenizer 138, or combinations thereof. The fractional handpiece may also include a vibrator 136, buzzer, mechano-oscillator, or sound system to warn an operator if there is a malfunction of the laser, such as laser missing fire. In additional examples, the fractional handpiece 16 may further include an attenuator 134 to achieve appropriate energy for the treatment of different skin types or LIOB depth. The attenuator may a neutral density filter or polarization-based elements (such as polarizer cube). The photodetector 132, the vibrator 136, the roller 118, the pump source laser 12, and/or the beam-splitting assembly 20 (e.g., the scanning mirrors 904) may be operatively connected to the controller 22, as described above. In one example, the two-dimensional microbeams are directed to the treatment site without the need for any guided rollers. In this case, the treatment operates in stamping mode. In another scenario, the handpiece may incorporate one or two rollers (912) to guide its movement. In this configuration, the rollers may serve to trigger the laser firing and synchronize with the galvo mirrors (904), as shown in FIG. 10C. The components of the fractional handpiece may be small enough to be packed into the handpiece 16. Such handpiece 16 may be operable to generate a picosecond laser beam, allowing for fractional treatment for skin rejuvenation.

An optional second or higher harmonic generator 300 may be located in handpiece body 16. FIG. 10A shows an example of the handpiece with second harmonic generator 300. Passively Q-switched laser assembly 18 may emit a beam with wavelength of 1064 nm. When additional laser wavelength is required, the second harmonic generator 300 may be introduced into the laser beam path to generate an additional laser wavelength. Generally, other wavelength frequency multiplying devices may be arranged on a turret and used when required. The temperature of second harmonic crystal 304 may be controlled by controller 22 for achieving stable and optimized frequency conversion.

FIG. 10B shows an example of the handpiece 16 with the second harmonic generator 300. The second harmonic generator 300 (e.g., a frequency doubling assembly) may generate a third wavelength that is a second harmonic wavelength of the second wavelength. Each micro-beam may have an energy of at least 1 mJ at the third wavelength. The handpiece 16 may also include a back reflection blocker 340 and a collimating lens 342 between the passively Q-switched assembly 18 and the second harmonic generator 300. The second harmonic generation assembly 300 may include the frequency doubling crystal 304, the dichroic mirror 306, and the beam dump 308. Additionally, the handpiece 16 may include the attenuator 134 and the homogenizer 138 between the second harmonic generator 300 and the beam-splitting assembly 20. The collimating lens 342 may be used to collimate the Q-switched laser at the second wavelength. The beam-splitting assembly 20 may be used to produce a two-dimensional microbeam pattern on the treatment plane. The beam-splitting assembly 20 may be configured in either a sliding mode with one scanning mirror or a stamping mode enabled by two scanning mirrors.

The back reflection blocker 340 may block any scattering or reflection of the laser from any optical surfaces of downstream optics that might disrupt proper Q-switching operation. In some examples, the back reflection blocker 340 may be a pinhole that is sized to allow the laser out of the passively Q-switched laser assembly 18 (e.g., out of the resonator of the assembly 18) to pass through while effectively blocking any reflection or scattering back into the resonator cavity. In other examples, the back reflection blocker 340 may be a dichroic mirror designed for highly transmitting the laser out of the passively Q-switched laser assembly 18 at the second wavelength (e.g., out of the resonator of the assembly 18) while exhibiting high reflectance to any reflections or scattering at the third wavelength. The back reflection blocker 340 may be positioned so that its normal direction is at least 10 degrees relative to the laser propagation axis.

The second harmonic generator 300 may be used to convert the incoming fundamental laser at the second wavelength to the laser at the third wavelength through second harmonic generation. The second harmonic generator 300 may be oriented at an angle of approximately 5 degrees with respect to the laser propagation axis. The dichroic mirror 306 may be used for highly transmitting the laser converted by the second harmonic generator 300 while effectively reflecting the unconverted laser at the second wavelength and directing it towards the beam dump 308. The attenuator 134 may be used to ensure that the delivered energy is in good agreement with the set value. The homogenizer 138 may be used to address variations in beam characteristics at different repetition rates and to ensure a uniform beam profile at the treatment plane. The homogenizer 138 may be diffractive-based or refractive-based optics.

FIG. 10C shows an example of the handpiece 16 with the second harmonic generator 300. The handpiece 16 may include the back reflection blocker 340 and the collimating lens 342 between the passively Q-switched assembly 18 and the second harmonic generator 300. The second harmonic generation assembly 300 may also include the frequency doubling crystal 304, the dichroic mirror 306, and the beam dump 308. Additionally, the handpiece 16 may include the homogenizer 138 between the second harmonic generator 300 and the beam-splitting assembly 20. Thus, the handpiece 16 shown in FIG. 10C may differ from that shown in FIG. 10B in that the laser energy is adjusted at the third wavelength by an alternative method to an attenuator. The frequency doubling crystal 304 may have a wedged surface of no less than 3 degree.

The second harmonic generator 300 may be rotational along a direction of an incoming laser beam at the second wavelength. Rotation of the frequency doubling crystal 304 of the second harmonic generator 300 may control or attenuate energy along the direction of the incoming laser beam at the third wavelength. The frequency doubling crystal 304 may be mounted to a rotational stage to enable rotation along the beam propagation axis. Maximum energy delivery occurs when the frequency doubling crystal 304 is oriented to meet the phase matching condition. Deviating from this optimal orientation through rotation results in reduced energy. Specifically, rotation of the frequency doubling crystal 304 from the optimal phase matching position to a 45-degree angle may shift laser energy from a maximum to a minimum or near-zero level for a type II phase matching. Additionally, rotation of the of the frequency doubling crystal 304 from the optimal phase matching position to a 90-degree angle may shift laser energy from a maximum to a minimum or near-zero level for a type I phase matching.

FIG. 10D shows an example of the handpiece 16 with the second harmonic generator 300. The handpiece 16 may include the back reflection blocker 340, the collimating lens 342, and a half-waveplate 344 between the passively Q-switched assembly 18 and the second harmonic generator 300. The second harmonic generation assembly 300 may include the frequency doubling crystal 304, the dichroic mirror 306, and the beam dump 308. Additionally, the handpiece 16 may include the homogenizer 138 between the second harmonic generator 300 and the beam-splitting assembly 20. Thus, the handpiece 16 shown in FIG. 10D may differ from that shown in FIG. 10B in that the laser energy is adjusted at the third wavelength by an alternative method to an attenuator.

The half-waveplate 344 may be rotational along a beam propagation direction, such that aligning the rotation of the half-waveplate 344 with the phase matching condition of the second harmonic generator 300 may maximize the laser energy at the third wavelength. Rotation of the half-waveplate 344 may control or attenuate energy at the third wavelength. Maximum energy delivery occurs when the half-waveplate 344 is oriented to meet the phase matching condition. Deviating from this optimal orientation through rotation results in reduced energy. Specifically, rotation of the half-waveplate 344 from the optimal phase matching position to a 22.5-degree angle may shift laser energy from a maximum to a minimum or near-zero level for a type II phase matching. Additionally, rotation of the half-waveplate 344 from the optimal phase matching position to a 45-degree angle may shift laser energy from a maximum to a minimum or near-zero level for a type I phase matching. FIG. 10T shows the relationship between half-waveplate rotation and pulse energy.

FIG. 10E shows an example of the handpiece 16 with the second harmonic generator 300 and a wavelength switching assembly 350 to enable selective delivery of two laser wavelengths (e.g., the second and/or third wavelength) within a single handpiece. Additionally, the handpiece 16 may adjust energy without the need for extra components. The handpiece 16 may include the back reflection blocker 340 and the collimating lens 342 between the passively Q-switched laser assembly 18 and the second harmonic generator 300. Additionally, the handpiece 16 may include the homogenizer 138 between the wavelength switching assembly 350 and the beam-splitting assembly 20. The second harmonic generator 300 may deliver the lasers at the second and third wavelengths simultaneously and collinearly.

The second harmonic generation assembly 300 may simultaneously deliver both the frequency-double laser and the fundamental laser at the second wavelength. Additionally, the second harmonic generation assembly 300 may facilitate energy adjustment for the lasers at these two wavelengths. By optimizing the phase matching condition, the second harmonic generation assembly 300 may facilitate maximizing laser energy at the third wavelength while minimizing unconverted fundamental laser energy. By adjusting the frequency doubling crystal 352 or the half waveplate 354 to minimize conversion efficiency, the lowest energy at the third wavelength may be achieved to result in the highest energy of unconverted laser at the second wavelength. Energy levels between these maximum and minimum values for the two wavelengths may be attained by adjusting the conversion efficiency of the second harmonic generation assembly 300 to an intermediate point between the two extremes.

As shown in FIG. 10F, the second harmonic generation assembly 300 may include a half-waveplate 354 for the second wavelength and the frequency doubling crystal 352. The half-waveplate 354 may be rotational to adjust the polarization of the fundamental laser, allowing tunability of the output energy at the two wavelengths based on the degree of the phase-matching condition. Rotation of the half-waveplate 354 may control or attenuate energy at the second and/or third wavelength. Maximum energy at the third wavelength delivery occurs when the half-waveplate 354 is oriented to meet the phase matching condition. Deviating from this optimal orientation through rotation results in reduced energy at the third wavelength. Specifically, rotation of the half-waveplate 344 from the optimal phase matching position to a 22.5-degree angle may shift laser energy at the third wavelength from a maximum to a minimum or near-zero level, while tuning laser energy at the second wavelength from a minimum to a maximum, for a type II phase matching. Additionally, rotation of the half-waveplate 354 from the optimal phase matching position to a 45-degree angle may shift laser energy at the third wavelength from a maximum to a minimum or near-zero level, while tuning laser energy at the second wavelength from a minimum to a maximum, for a type I phase matching.

As shown in FIG. 10G, the second harmonic generation assembly 300 may include the frequency doubling crystal 352. The frequency doubling crystal 352 may be rotational to adjust the crystal orientation relative to the polarization of the fundamental laser, allowing tunability of the output energy at the two wavelengths based on the degree of the phase-matching condition. Rotation of the frequency doubling crystal 352 may occur along a direction of an incoming laser beam at the second wavelength.

Rotation of the frequency doubling crystal 352 may control or attenuate energy at the second and/or third wavelength. Maximum energy delivery at the third wavelength occurs when the frequency doubling crystal 352 is oriented to meet the phase matching condition. Deviating from this optimal orientation through rotation results in reduced energy at the third wavelength. Specifically, rotation of the frequency doubling crystal 352 from the optimal phase matching position to a 45-degree angle may shift laser energy at the third wavelength from a maximum to a minimum or near-zero level, while tuning laser energy at the second wavelength from a minimum to a maximum, for a type II phase matching. Additionally, rotation of the frequency doubling crystal 352 from the optimal phase matching position to a 90-degree angle may shift laser energy at the third wavelength from a maximum to a minimum or near-zero level, while tuning laser energy at the second wavelength from a minimum to a maximum, for a type I phase matching.

The wavelength switching assembly 350 may comprise a set of optical components essential for switching between the second and third wavelengths. As shown in FIGS. 10H-10Q, The selection of the second wavelength involves redirecting the laser emitting at the third wavelength into a beam dump or halting the laser propagation at the third wavelength using a shutter. Similarly, choosing the third wavelength entails redirecting the laser emitting at the second wavelength into the beam dump or blocking the laser at the second wavelength with a shutter.

As shown in FIGS. 10H-10I, the wavelength switching assembly 350 may include a pair of dichroic mirrors 356 and a beam dump 358. The selective positioning of the dichroic mirrors 356 may be implemented by rotating the dichroic mirrors 356 along their symmetry axis. As shown in FIG. 10H, the dichroic mirror 356a positioned in the beam path may have coatings that are highly reflective at the third wavelength and highly transmissive at the second wavelength. As shown in FIG. 10I, the dichroic mirror 356b positioned in the beam path may have coatings that are highly reflective at the second wavelength and highly transmissive at the third wavelength. Thus, the wavelength switching assembly 350 will deliver the second wavelength when the dichroic mirror 356a is placed in the beam path and the third wavelength when the dichroic mirror 356b is placed in the beam path.

As shown in FIGS. 10J-10K, the wavelength switching assembly 350 may include the pair of dichroic mirrors 356 and the beam dump 358. The selective positioning of the dichroic mirrors 356 may be implemented by translating the dichroic mirrors 356. As shown in FIG. 10J, the dichroic mirror 356a positioned in the beam path may have coatings that are highly reflective at the third wavelength and highly transmissive at the second wavelength. As shown in FIG. 10K, the dichroic mirror 356b positioned in the beam path may have coatings that are highly reflective at the second wavelength and highly transmissive at the third wavelength. Thus, the wavelength switching assembly 350 will deliver the second wavelength when the dichroic mirror 356a is placed in the beam path and the third wavelength when the dichroic mirror 356b is placed in the beam path.

As shown in FIGS. 10L-10M, the wavelength switching assembly 350 may include a moveable dichroic mirror 360 and the beam dump 358. The moveable dichroic mirror 360 may include two mirror segments 360a and 360b, each with a different coating, as shown in FIGS. 10R-10S. The moveable dichroic mirror 360 can have a rectangular, oval, circular, or other shape. As shown in FIG. 10L, the mirror segment 360a positioned in the beam path may have a coating that is highly transmissive at the second or third wavelength and highly reflective at the other wavelength to selectively deliver one of the wavelengths to skin. As shown in FIG. 10M, the mirror segment 360b positioned in the beam path may have a coating that is highly reflective at the second or third wavelength and highly transmissive at the other wavelength to selectively deliver one of the wavelengths to skin. Thus, the wavelength switching assembly 350 will deliver the second wavelength when the mirror segment 360a is placed in the beam path and the third wavelength when the mirror segment 360b is placed in the beam path. The positioning of the mirror segments 360a or 360b into the beam path may be implemented by translation or rotation, e.g., the movable dichroic mirror 360 may be translated or rotated. The movement can be manually or automatically driven by a motor. The movable dichroic mirror 360 can be manufactured using commonly-known approaches which typically consists of multiple thin layers of dielectric materials sputtered, e.g., electron beam deposition, ion beam sputtering (IBS) or ion-assisted deposition, onto a substrate such as BK7 glass. The substrate may be an optical window, a mirror blanks, semiconductor materials, or other materials known in the field.

As shown in FIGS. 10N-10O, the wavelength switching assembly 350 may include a first dichroic mirror 362, a second dichroic mirror 363, two mirrors 364, and the beam dump 358. The dichroic mirror 362 separates the two lasers at the second and third wavelengths into two beam paths. The wavelength selection by the wavelength switching assembly 350 may be implemented by moving the second dichroic mirror 363 in and out of the beam path. The second dichroic mirror 363 may be translational or flippable. The two mirrors 364 are highly reflective at the third wavelength. As shown in FIGS. 10N-10O, the first dichroic mirror 363 has coatings that are highly reflective at the third wavelength and highly transmissive at the second wavelength. As shown in FIG. 10O, the second dichroic mirror 363 has coatings that are highly reflective at the second and third wavelength. Thus, the wavelength switching assembly 350 will deliver the second wavelength when the second dichroic mirror 363 is moved out of the beam path and the third wavelength when the second dichroic mirror 363 is moved in the beam path.

As shown in FIGS. 10P-10Q, the wavelength switching assembly 350 may include the first dichroic mirror 362, the second dichroic mirror 363, two mirrors 364, a first shutter 366, and a second shutter 368. The dichroic mirror 362 separates the two lasers at the second and third wavelengths into two beam paths. The wavelength selection by the wavelength switching assembly 350 may be implemented by moving the first and second shutters 366, 368 in and out of their corresponding beam paths. The first and second dichroic mirrors 362 and 363 have coatings that are highly reflective at the third wavelength and highly transmissive at the second wavelength. The two mirrors 364 are highly reflective at the third wavelength. The wavelength switching assembly 350 will deliver the second wavelength when the first shutter 366 is moved out of the second wavelength beam path while the second shutter 368 is positioned in the third wavelength beam path. The wavelength switching assembly 350 will deliver the third wavelength when the second shutter 368 is moved out of the third wavelength beam path and the first shutter 366 is positioned in the second wavelength beam path.

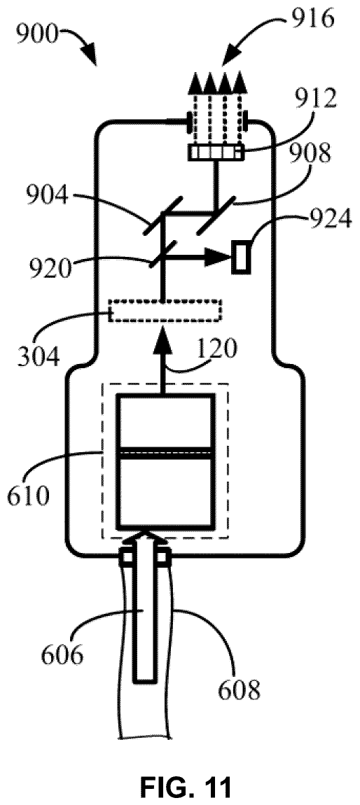

In a further example illustrated in FIG. 11, a handpiece for fractional skin treatment is shown, where the passively Q-switched laser assembly includes a monolithic cavity 610. Generation of a fractionated beam pattern is produced by a combination of a pair of galvo mirrors 904 and 908 that project or scan laser beam 120 onto a lens array 912. Lens array 912 splits laser beam 120 into a plurality of micro-beams 916 which may be fractionated micro-beams. Mirror 920 may be used to separate additional wavelengths generated by the second or higher harmonic generator 304. The coating of mirror 920 is formed accordingly to the desired wavelength separation. The unconverted infrared light 120 may be directed and absorbed in a laser light beam dump 924 while the harmonics may be delivered to the treated skin segment containing a combination of skin disorders. Laser light beam dump 924 may be operable to effectively dissipate the unconverted infrared energy without getting damaged or causing a rise in temperature of other handpiece 900 components. Passive and active cooling mechanisms can be used as needed to remove heat from the laser light beam dump 924.

The example below provides some operational parameters of a typical handpiece used for skin disorder treatment. Energy for the output laser 120 of the passively Q-switched laser assembly may be 40 mJ or more. For example, energy for each micro-beam 122 may be up to 4 mJ at 1064 nm and up to 2 mJ at 532 nm.

Such laser energy is sufficiently high to allow each laser beam from the passively Q-switched laser assembly to be split into 10 microbeams using a 1D beamsplitter, with each microbeam containing at least 4 mJ, as illustrated in FIG. 7B. In an alternative example, a laser beam of at least 40 mJ from the passively Q-switched laser assembly can cover at least 9 lenslets of a microlens array to produce 9 micro-dots, as shown in FIG. 7D-7E. Galvo mirror pair 904 and 908 scans the laser beam nine times to form a 2-D pattern and cover at least 81 lenslets. Assuming that microchip laser operates at a frequency of 20 Hz, each scan will take 0.45 seconds (9/20) or the treatment can be operated up to 2.2 Hz.

In another example, the energy out of the laser 120 of the passively Q-switched laser assembly may be about or below 4 mJ, 3 mJ, 2 mJ, or 1 mJ which is sufficient for a single microbeam to achieve adequate treatment. In this case, the fractionated beam pattern has to rely on the scanning of the laser beam out of the passively Q-switched laser with at least one scanning mirror followed by beam focusing by a focusing lens.

Further provided herein are methods for skin treatment. The method may include delivering a sub-nanosecond pulsed laser beam to a patient in need thereof using the laser system with a fractional handpiece.

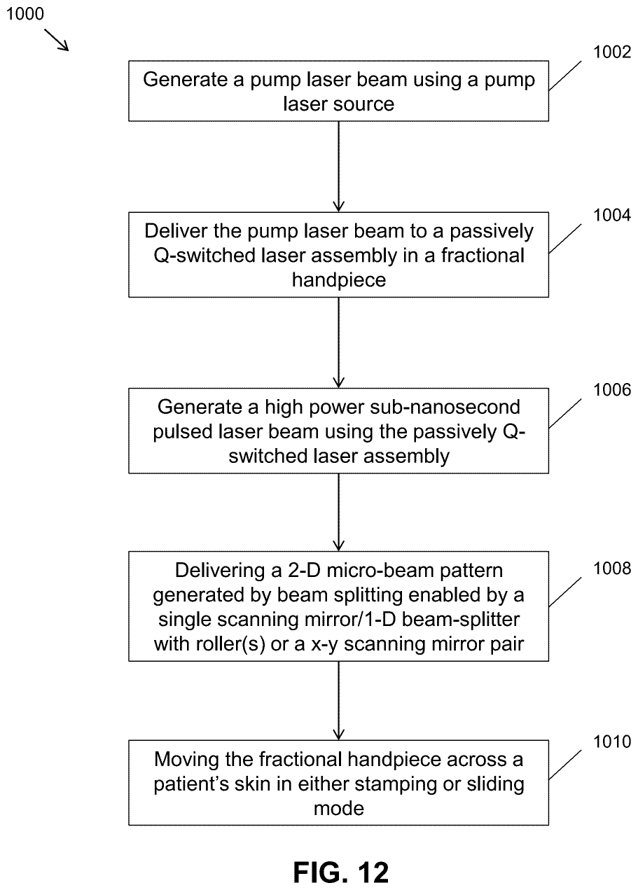

Referring to FIG. 12, a flowchart is presented in accordance with an example embodiment. The method 1000 is provided by way of example, as there are a variety of ways to carry out the method. The method 1000 described below can be carried out using the configurations illustrated in FIGS. 1-11, for example, and various elements of these figures are referenced in explaining example method 1000. Each block shown in FIG. 12 represents one or more processes, methods or subroutines, carried out in the example method 1000. Furthermore, the illustrated order of blocks is illustrative only and the order of the blocks can change according to the present disclosure. Additional blocks may be added, or fewer blocks may be utilized, without departing from this disclosure.

The example method 1000 is a method for skin treatment in a patient in need thereof. The example method 1000 can begin at block 1002. At block 1002, a pump laser source generates a pump laser beam at a first wavelength. For example, for an Nd:YAG laser, the pump laser wavelength may be within one of three wavelength bands, for example, 735-760 nm, 795-820 nm, or 865-885 nm. The pump laser may be a solid state laser or diode laser. Non-limiting examples of pump lasers include an Alexandrite laser (755 nm), a Ti:Sapphire laser, a diode laser, a dye laser, an optical parametric oscillator (OPO), and an optical parameter amplifier (OPA). Ti:Sapphire may be used to generate laser beams in the wavelength range between 700-900 nm via direct emission pumped in the visual wavelength region.

In a non-limiting example, a 755 nm wavelength pump laser beam may be generated from an Alexandrite laser.

At block 1004, the pump laser beam is delivered to a passively Q-switched laser assembly in a fractional handpiece. In other examples, the pump laser source may be located within the body of the fractional handpiece and the pump laser beam may directly illuminate the passively Q-switched laser assembly.

At block 1006, the passively Q-switched laser assembly in the fractional handpiece generates a high-power sub-nanosecond pulsed laser beam in the near infrared region pumped by the pump laser beam. In an example, the generated sub-nanosecond pulsed laser beam has a second wavelength. For Nd:YAG gain material, a 1064 nm laser is generated. In another scenario using Nd:YLF laser material, wavelengths of 1.053 μm or 1.047 μm may be generated from a passively Q-switched Nd:YLF laser. Frequency doubling of such near-infrared lasers (around 1 μm) can produce lasers at visible wavelengths, where melanin exhibits substantial absorption. Some of non-limiting examples include 532 nm, 524 nm, or 528 nm. The sub-nanosecond pulsed laser beam may optionally be delivered to the fractional handpiece via a laser delivery unit.

At block 1008, the sub-nanosecond pulsed laser beam having the second wavelength is delivered to the treatment plane in a form of a 2-D micro-beam pattern generated by beam splitting enabled by a single scanning mirror or 1-D beam splitter with roller(s) or a x-y scanning mirror pair.

At block 1010, the fractional handpiece is moved across the skin in either stamping or sliding mode. The delivered laser beam may be applied to a target area of the patient's skin. The target area may be on any area of the patient's skin, including but not limited to the face, arm, leg, back, chest, hand, or foot.

It will be appreciated by persons skilled in the art that the present disclosure is not limited to what has been particularly shown and described hereinabove. Rather, the scope of the passively Q-switched laser and handpiece includes both combinations and sub-combinations of various features described hereinabove as well as modifications and variations thereof which would occur to a person skilled in the art upon reading the foregoing description and which are not in the prior art.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

Numerous examples are provided herein to enhance the understanding of the present disclosure. A specific set of statements are provided as follows.

Statement 1: A fractional handpiece for skin treatment comprising: a handpiece body comprising: a passively Q-switched laser assembly within the handpiece body operatively connected to a pump laser source to receive a pump laser beam having a first wavelength; and a beam-splitting assembly operable to scan a solid beam emitted by the passively Q-switched laser assembly and form micro-beams across a segment of skin; and wherein the passively Q-switched laser assembly generates a sub-nanosecond pulsed laser beam having a second wavelength; wherein the passively Q-switched laser assembly has an unstable cavity operation.

Statement 2: The fractional handpiece of statement 1, wherein a repetition rate of the beam-splitting assembly is about 100 pulses per second to about 500 pulses per second.

Statement 3: The fractional handpiece of statement 1, wherein a cavity length of the passively Q-switched laser assembly is less than about 10 mm.

Statement 4: The fractional handpiece of statement 1, wherein the passively Q-switched laser assembly comprises a plano high reflector at a proximal end, a rare earth ion-doped gain material, a saturable absorber, and a convex output coupler at a distal end of the passively Q-switched laser assembly.

Statement 5: The fractional handpiece of statement 1, wherein the passively Q-switched laser assembly comprises a convex high reflector at a proximal end, a rare earth ion-doped gain material, a saturable absorber, and a plano output coupler at a distal end of the passively Q-switched laser assembly.

Statement 6: The fractional handpiece of statement 1, wherein the passively Q-switched laser assembly comprises a bonded element, in which the rare earth ion-doped gain material is bonded with an undoped material at a proximal end of the gain material, which is transparent at the first and second wavelengths.

Statement 7: The fractional handpiece of statement 6, wherein the undoped transparent material is the same material as the host material of the rare earth ion-doped gain material or a different material from the host material of the rare earth ion-doped gain material.

Statement 8: The fractional handpiece of statement 6, wherein a resonator is formed by the convex high reflector and a distal end of the saturable absorber coated with a partial reflection coating at the second wavelength.

Statement 9: The fractional handpiece of statement 6, wherein the proximal end of the undoped transparent material has a concave curvature and is coated with a highly reflecting coating at the second wavelength and a highly transmitting coating at the first wavelength to act as a high reflector, and a plano mirror serves as an output coupler.

Statement 10: The fractional handpiece of statement 6, wherein a resonator is formed by a convex output coupler and the plano proximal end of the undoped transparent material coated with a highly reflecting coating at the second wavelength and a highly transmitting coating at the first wavelength.

Statement 11: The fractional handpiece of statement 6, wherein either the convex high reflector or the convex output coupler has a convex curvature facing the resonator and the proximal end of the undoped transparent material is a plano surface while another external plano serves as an output coupler or a high reflector.

Statement 12: The fractional handpiece of statement 1, wherein a proximal end of a gain medium is bonded with an undoped transparent material and the distal end of the gain medium is bonded with a saturable absorber to form a monolithic element.

Statement 13: The fractional handpiece of statement 12, wherein the undoped transparent material has a concave curvature on its entrance surface and has coatings to highly reflect the laser beam at the second wavelength and highly transmit the laser beam at the first wavelength, and wherein a distal end of the saturable absorber has a partially reflecting coating at the second wavelength.

Statement 14: The fractional handpiece of statement 13, wherein parallelism between a proximal end of the undoped transparent material and the distal end of the saturable absorber is within 10 arc seconds.

Statement 15: The fractional handpiece of statement 12, wherein a resonator is formed by a convex high reflector and the distal end of the saturable absorber, the distal end of the saturable absorber being coated with a partially reflecting coating at the second wavelength.

Statement 16: The fractional handpiece of statement 12, wherein a resonator is formed by a convex output coupler and a proximal end of the undoped transparent material, the proximal end of the undoped transparent material being coated with a highly reflecting coating at the second wavelength and a highly transmitting coating at the first wavelength.

Statement 17: The fractional handpiece of statement 1, wherein the pulsed laser beam at the second wavelength has an energy of no less than 2 mJ.

Statement 18: The fractional handpiece of statement 1, wherein the fractional handpiece does not have an amplifier.

Statement 19: The fractional handpiece of statement 1, further comprising a homogenizer after the passively Q-switched laser assembly to mitigate beam characteristic variation at different repetition rates, and to homogenize a beam profile delivered to the skin.

Statement 20: The fractional handpiece of statement 1, wherein the unstable cavity operation improves beam quality and beam mode stability, and stabilizes pulse duration of the pulsed laser beam at the second wavelength.

Statement 21: The fractional handpiece of statement 1, wherein the pump laser source is a diode laser operable to emit the first wavelength at about 750 nm to about 980 nm.

Statement 22: The fractional handpiece of statement 1, wherein the pump laser source operates from a single pulse to a frequency up to about 2000 Hz.

Statement 23: The fractional handpiece of statement 1, wherein the beam-splitting assembly consists of one or more rollers and a scanning mirror, capable of generating a single line of micro-dots.

Statement 24: The fractional handpiece of statement 23, wherein the one or more rollers are on a tip of the fractional handpiece to guide movement of the fractional handpiece and to synchronize with laser pulsing and the beam-splitting assembly.

Statement 25: The fractional handpiece of statement 24, wherein the tip is disposable.

Statement 26: The fractional handpiece of statement 1, wherein the beam-splitting assembly comprises two scanning mirrors which can scan the solid beam along two perpendicular directions, creating a two-dimensional micro-beam pattern.

Statement 27: The fractional handpiece of statement 26, wherein microdot surface coverage and density can be adjusted by programming control of the two scanning mirrors.

Statement 28: The fractional handpiece of statement 1, further comprising a frequency doubling assembly comprising a frequency doubling crystal to deliver a third wavelength at a second harmonic wavelength of the second wavelength.

Statement 29: The fractional handpiece of statement 28, wherein each micro-beam has an energy of at least 1 mJ at the third wavelength.

Statement 30: The fractional handpiece of statement 28, wherein a frequency doubling crystal is rotational along a direction of an incoming laser beam at the second wavelength.

Statement 31: The fractional handpiece of statement 30, wherein energy control or attenuation at the third wavelength is implemented by rotating the frequency doubling crystal along the direction of the incoming laser beam at the second wavelength.

Statement 32: The fractional handpiece of statement 31, wherein rotating the frequency doubling crystal from this optimal phase matching position to a 45-degree angle shifts laser energy from a maximum to a minimum or near-zero level for a type II phase matching.

Statement 33: The fractional handpiece of statement 31, wherein rotating the frequency doubling crystal from this optimal phase matching position to a 90-degree angle shifts laser energy from a maximum to a minimum or near-zero level for a type I phase matching.

Statement 34: The fractional handpiece of statement 28, wherein a half waveplate is positioned immediately before the frequency doubling assembly and can be rotated along a beam propagation direction.

Statement 35: The fractional handpiece of statement 34, wherein a rotation of the half waveplate is used to control or attenuate laser energy at the third wavelength.

Statement 36: The fractional handpiece of statement 35, wherein rotating the half waveplate from this optimal phase matching position to a 22.5-degree angle shifts the laser energy from a maximum to a minimum or near-zero level for a type II phase matching.

Statement 37: The fractional handpiece of statement 35, wherein rotating the half waveplate from this optimal phase matching position to a 45-degree angle shifts the laser energy from a maximum to a minimum or near-zero level for a type I phase matching.

Statement 38: The fractional handpiece of statement 28, wherein the frequency doubling crystal has a wedged surface of no less than 3 degree.

Statement 39: The fractional handpiece of statement 28, wherein a dichroic mirror is placed between the passively Q-switched laser assembly and the frequency doubling crystal to highly transmit a laser at the second wavelength and highly reflect any scattering or reflection of the laser at the third wavelength back to the passively Q-switched laser assembly.

Statement 40: The fractional handpiece of statement 28, wherein a pinhole is positioned between the passively Q-switched laser assembly and the frequency doubling crystal to fully pass a laser at the second wavelength but block the reflection from a proximal end of the frequency-doubling crystal at both the second and third wavelengths.

Statement 41: A fractional handpiece for skin treatment comprising: a handpiece body comprising: a passively Q-switched laser assembly within the handpiece body operatively connected to a pump laser source to receive a pump laser beam having a first wavelength to generate a sub-nanosecond pulsed laser beam having a second wavelength; a second harmonic generation assembly operatively delivering lasers with variable energies at the second and third wavelengths, the third wavelength being a second harmonic of the second wavelength; a wavelength switching assembly selectively delivering either the second wavelength or the third wavelength; and a beam-splitting assembly operable to scan a solid beam emitted by the passively Q-switched laser assembly and form micro-beams across a segment of skin; and wherein the passively Q-switched laser assembly has an unstable cavity operation.

Statement 42: The fractional handpiece of statement 41, wherein the second harmonic generation assembly can deliver the lasers at the second and third wavelengths simultaneously and collinearly.

Statement 43: The fractional handpiece of statement 41, wherein the second harmonic generation assembly comprises a rotational second harmonic crystal.

Statement 44: The fractional handpiece of statement 43, wherein a frequency doubling crystal is rotational along a direction of an incoming laser beam at the second wavelength.

Statement 45: The fractional handpiece of statement 44, wherein rotating the frequency doubling crystal from this optimal phase matching position to a 45-degree angle shifts laser energy at the third wavelength from a maximum to a minimum or near-zero level while tuning laser energy at the second wavelength from a minimum to a maximum, specifically for a type II phase matching.

Statement 46: The fractional handpiece of statement 44, wherein rotating the frequency doubling crystal from this optimal phase matching position to a 90-degree angle shifts laser energy at the third wavelength from a maximum to a minimum or near-zero level while tuning laser energy at the second wavelength from a minimum to a maximum, specifically for a type I phase matching.

Statement 47: The fractional handpiece of statement 41, wherein the second harmonic generation assembly comprises a rotational half waveplate for the second wavelength immediately before a second harmonic crystal.

Statement 48: The fractional handpiece of statement 47, wherein rotating the half waveplate from this optimal phase matching position to a 22.5-degree angle shifts laser energy at the third wavelength from a maximum to a minimum or near-zero level while tuning laser energy at the second wavelength from a minimum to a maximum, specifically for a type II phase matching.

Statement 49: The fractional handpiece of statement 47, wherein rotating the half waveplate from this optimal phase matching position to a 45-degree angle shifts laser energy at the third wavelength from a maximum to a minimum or near-zero level while tuning laser energy at the second wavelength from a minimum to a maximum, specifically for a type I phase matching.

Statement 50: The fractional handpiece of statement 41, wherein the wavelength switching assembly comprises two dichroic mirrors which can be selectively positioned in a beam path via rotation along a symmetry axis of the two dichroic mirrors, allowing the choice of either the second wavelength or the third wavelength as an output wavelength.

Statement 51: The fractional handpiece of statement 50, wherein one dichroic mirror has coatings which highly transmit the laser at the second wavelength and highly reflect the laser at the third wavelength while the other dichroic mirror has coatings which is highly reflective at the second wavelength and highly transmissive at the third wavelength.

Statement 52: The fractional handpiece of statement 41, wherein the wavelength switching assembly comprises two dichroic mirrors which can be selectively positioned in a beam path via translation to choose either the second wavelength or the third wavelength as an output wavelength.

Statement 53: The fractional handpiece of statement 52, wherein one dichroic mirror has coatings which is highly transmissive at the second wavelength and highly reflective at the third wavelength while the other dichroic mirror has coatings which highly reflects the laser at the second wavelength and highly transmits the laser at the third wavelength.

Statement 54: The fractional handpiece of statement 41, wherein the lasers at the second and third wavelengths from the second harmonic generation assembly are separated into two beam paths by a dichroic mirror, which has coatings to highly reflect the lasers at one wavelength and highly transmit the lasers at the other wavelength.

Statement 55: The fractional handpiece of statement 54, wherein a second dichroic mirror can be moved in and out of a beam path of the lasers to selectively deliver a target wavelength as an output.

Statement 56: The fractional handpiece of statement 54, wherein a second dichroic mirror has highly reflective coatings at the second and third wavelengths.

Statement 57: The fractional handpiece of statement 54, wherein two shutters can be selectively positioned in or out of a beam path of the lasers to select a corresponding laser at a desirable wavelength, which can be directed to the beam-splitting assembly by a second dichroic mirror.

Statement 58: The fractional handpiece of statement 57, wherein the second dichroic mirror is coated to highly reflect the lasers at the second or third wavelengths, while allowing high transmission of the lasers at the other wavelength.

Statement 59: The fractional handpiece of statement 41, wherein the wavelength switching assembly comprises a movable mirror consisting of two segments coated with two different types of dichroic coatings.

Statement 60: The fractional handpiece of statement 59, wherein one segment features coatings highly transmitting at the second wavelength and highly reflective at the third wavelength, while the other segment has coatings that highly reflect the laser at the second wavelength and highly transmit the laser at the third wavelength.

Statement 61: The fractional handpiece of statement 59, wherein the two segments of the movable mirror can be selectively positioned in the beam path, enabling the direction of the laser at the targeted wavelength to the beam-splitting assembly as the final output through either rotation or translation.

1. A fractional handpiece for skin treatment comprising:
a handpiece body comprising:
a passively Q-switched laser assembly within the handpiece body operatively connected to a pump laser source to receive a pump laser beam having a first wavelength; and
a beam-splitting assembly operable to scan a solid beam emitted by the passively Q-switched laser assembly and form micro-beams across a segment of skin,
wherein the passively Q-switched laser assembly generates a sub-nanosecond pulsed laser beam having a second wavelength, and
wherein the passively Q-switched laser assembly has an unstable cavity operation.

2. The fractional handpiece of claim 1, wherein a repetition rate of the beam-splitting assembly is about 100 pulses per second to about 500 pulses per second.

3. The fractional handpiece of claim 1, wherein a cavity length of the passively Q-switched laser assembly is less than about 10 mm.

4. The fractional handpiece of claim 1, wherein the passively Q-switched laser assembly comprises either a convex high reflector or a convex output coupler having a convex curvature facing a resonator, a rare earth ion-doped gain material, a saturable absorber and another external plano mirror serving as an output coupler or a high reflector, and wherein the passively Q-switched laser assembly comprises a bonded element, in which the rare earth ion-doped gain material is bonded with an undoped material at a proximal end of the gain material, which is transparent at the first and second wavelengths

5. (canceled)

6. The fractional handpiece of claim 4, wherein a resonator is formed by the convex high reflector and a distal end of the saturable absorber coated with a partial reflection coating at the second wavelength.

7. The fractional handpiece of claim 4, wherein the proximal end of the undoped transparent material has a concave curvature and is coated with a highly reflecting coating at the second wavelength and a highly transmitting coating at the first wavelength to act as a high reflector, and a plano mirror serves as an output coupler.

8. The fractional handpiece of claim 4, wherein a resonator is formed by a convex output coupler and the plano proximal end of the undoped transparent material coated with a highly reflecting coating at the second wavelength and a highly transmitting coating at the first wavelength.

9. The fractional handpiece of claim 4, wherein either the convex high reflector or the convex output coupler has a convex curvature facing the resonator and the proximal end of the undoped transparent material is a plano surface while another external plano serves as an output coupler or a high reflector.

10. The fractional handpiece of claim 1, wherein a proximal end of a gain medium is bonded with an undoped transparent material and a distal end of the gain medium is bonded with a saturable absorber to form a monolithic element, and wherein the undoped transparent material has a concave curvature on its entrance surface and has coatings to highly reflect the laser beam at the second wavelength and highly transmit the laser beam at the first wavelength, and wherein a distal end of the saturable absorber has a partially reflecting coating at the second wavelength.

11. (canceled)

12. The fractional handpiece of claim 10, wherein parallelism between a proximal end of the undoped transparent material and the distal end of the saturable absorber is within 10 arc seconds.

13. The fractional handpiece of claim 10, wherein either a convex high reflector or a convex output coupler has a convex curvature facing the monolithic element and the proximal end of the undoped transparent material is a plano surface while another external plano surface serves as an output coupler or a high reflector.

14. (canceled)

15. (canceled)

16. The fractional handpiece of claim 1, further comprising a homogenizer after the passively Q-switched laser assembly to mitigate beam characteristic variation at different repetition rates, and to homogenize a beam profile delivered to the skin.

17. The fractional handpiece of claim 1, wherein the unstable cavity operation improves beam quality and beam mode stability and stabilizes pulse duration of the pulsed laser beam at the second wavelength.

18. (canceled)

19. (canceled)

20. The fractional handpiece of claim 1, wherein the beam-splitting assembly consists of one or more rollers and a scanning mirror, capable of generating a single line of micro-dots, and wherein the one or more rollers are on a tip of the fractional handpiece to guide movement of the fractional handpiece and to synchronize with laser pulsing and the beam-splitting assembly.

21. (canceled)

22. The fractional handpiece of claim 1, wherein the beam-splitting assembly comprises two scanning mirrors which can scan the solid beam along two perpendicular directions, creating a two-dimensional micro-beam pattern, and wherein microdot surface coverage and density can be adjusted by programming control of the two scanning mirrors.

23. (canceled)

24. A fractional handpiece for skin treatment comprising:
a handpiece body comprising:
a passively Q-switched laser assembly within the handpiece body operatively connected to a pump laser source to receive a pump laser beam having a first wavelength to generate a sub-nanosecond pulsed laser beam having a second wavelength;
a second harmonic generation assembly operatively delivering lasers with variable energies at the second wavelength and a third wavelength, the third wavelength being a second harmonic of the second wavelength; and
a beam-splitting assembly operable to scan a solid beam emitted by the passively Q-switched laser assembly and form micro-beams across a segment of skin,
wherein the passively Q-switched laser assembly has an unstable cavity operation.

25. The fractional handpiece of claim 24, further comprising a frequency doubling assembly comprising a frequency doubling crystal to deliver a third wavelength at a second harmonic wavelength of the second wavelength, and wherein each micro-beam has an energy of at least 1 mJ at the third wavelength.

26. (canceled)

27. The fractional handpiece of claim 24, wherein a frequency doubling crystal is rotational along a direction of an incoming laser beam at the second wavelength.

28. A fractional handpiece for skin treatment comprising:
a handpiece body comprising:
a passively Q-switched laser assembly within the handpiece body operatively connected to a pump laser source to receive a pump laser beam having a first wavelength to generate a sub-nanosecond pulsed laser beam having a second wavelength;
a second harmonic generation assembly operatively delivering lasers with variable energies at the second wavelength and a third wavelength, the third wavelength being a second harmonic of the second wavelength;
a wavelength switching assembly selectively delivering either the second wavelength or the third wavelength; and
a beam-splitting assembly operable to scan a solid beam emitted by the passively Q-switched laser assembly and form micro-beams across a segment of skin,
wherein the passively Q-switched laser assembly has an unstable cavity operation.

29. The fractional handpiece of claim 28, wherein the wavelength switching assembly further consists of at least one moveable dichroic mirror with coatings highly reflective at the second wavelength or the third wavelength and highly transmissive at the other wavelength to selectively deliver one of the wavelengths to skin.

30. The fractional handpiece of claim 28, wherein the wavelength switching assembly comprises a movable mirror consisting of two segments coated with two different types of dichroic coatings to selectively deliver either the second wavelength or the third wavelength to skin.
